(19) United States
(12) Patent Application Publication
(10) Pub. No.: US 2006/0110753 A1
(43) Pub. Date: May 25, 2006

(54) Methods to diagnose and treat lung cancer

(76) Inventor: Bruce L. Roberts, Southboro, MA (US)

Correspondence Address: GENZYME CORPORATION;LEGAL DEPARTMENT, 15 PLEASANT ST CONNECTOR, FRAMINGHAM, MA, 01701-9322, US

(21) Appl. No.: 11/247,437

(22) Filed: Oct. 11, 2005

(30) Foreign Application Priority Data
Apr. 12, 2004 (WO) PCT/US04/11193

(51) Int. Cl. C12Q 1/68; G01N 33/574

(52) U.S. Cl. 435/6; 435/7.23

(57) ABSTRACT

The present invention provides compositions and methods for aiding in the diagnoses of the neoplastic condition of a lung cell, and methods of screening for a potential therapeutic agents for the reversal of the neoplastic condition. Also provided are therapeutic compositions and methods to inhibit the growth of neoplastic lung cells and to treat subjects harboring neoplastic lung cells.

### CROSS-REFERENCE TO RELATED APPLICATION

This application claims the benefit under 35 U.S.C. 119(d) of U.S. Provisional Application Ser. No. 60/462,028, filed Apr. 10, 2003, the contents of which are hereby incorporated by reference herein.

### TECHNICAL FIELD

This invention is in the field of cancer biology. In particular, the present invention provides compositions and methods for identifying a neoplastic lung cell. It also provides compositions and methods to inhibit the growth of neoplastic lung cells identified by these methods.

### BACKGROUND

Despite numerous advances in medical research, cancer remains the second leading cause of death in the United States. In the industrialized nations, roughly one in five persons will die of cancer. Traditional modes of clinical care, such as surgical resection, radiotherapy and chemotherapy, have a significant failure rate, especially for solid tumors. Failure occurs either because the initial tumor is unresponsive or because of recurrence due to regrowth at the original site and/or metastases.

Lung cancer is one of the most common malignancies worldwide and is the second leading cause of cancer death in man. See, American Cancer Society, Cancer Facts and Figures, 1996, Atlanta. Approximately 178,100 new cases of lung cancer were diagnosed in 1997, accounting for 13% of cancer diagnoses. An estimated 160,400 deaths due to lung cancer would occur in 1997 accounting for 29% of all cancer deaths. The one-year survival rates for lung cancer have increased from 32% in 1973 to 41% in 1993, largely due to improvements in surgical techniques. The 5 year survival rate for all stages combined is only 14%. The survival rate is 48% for cases detected when the disease is still localized, but only 15% of lung cancers are discovered that early. Among various forms of lung cancer, non-small cell lung cancer (NSCLC) accounts for nearly 80% of all new lung cancer cases each year. For patients diagnosed with NSCLC, surgical resection offers the only chance of meaningful survival. On the other hand, small cell lung cancer is the most malignant and fastest growing form of lung cancer and accounts for the rest of approximately 20% of new cases of lung cancer. The primary tumor is generally responsive to chemotherapy, but is followed by wide-spread metastasis. The median survival time at diagnosis is approximately 1 year, with a 5 year survival rate of 5%.

In spite of major advances in cancer therapy including improvements in surgical resection, radiation treatment and chemotherapy, successful intervention for lung cancer in particular, relies on early detection of the cancerous cells. Neoplasia resulting in benign tumors may be completely cured by removing the mass surgically. If a tumor becomes malignant, as manifested by invasion of surrounding tissue, it becomes much more difficult to eradicate. Therefore, there remains a considerable need in the art for the development of methods for detecting the disease at the early stage. There also exits a pressing need in the art for developing diagnostic methods to monitor or prognose the progression of the disease as well as methods to treat various related pathological conditions. This invention satisfies these needs and provides related advantages as well.

### DISCLOSURE OF THE INVENTION

The present invention provides methods for aiding in the diagnoses of the condition of a lung cell, for identifying and/or distinguishing normal and neoplastic lung cells and for identifying potential therapeutic agents to reverse neoplasia and/or ameliorate the symptoms associated with the presence of neoplastic lung cells in a subject. Further provided are compositions and methods to reverse neoplasia and/or ameliorate the symptoms associated with neoplastic lung cells in vivo.

Accordingly, one embodiment is a method of diagnosing the condition of a lung cell by screening for the presence of a differentially expressed gene isolated from a sample containing or suspected of containing a lung cell, in which the differential expression of the gene is indicative of the neoplastic state of the lung cell. In one aspect, the gene is expressed more in a neoplastic lung cell or a lung tumor cell as compared to normal lung cell, and is selected from EGFR-RS, RYK, TNFRSF25, TRPM7, KCP3 and KIAA 1883. In another aspect, the gene is expressed more in a normal lung cell as compared to a neoplastic lung cell, e.g., UNC5H2. In a yet further aspect, the gene was not heretofor known to be associated with lung cancer cells and therefore provides a diagnostic and prognostic marker as well as a therapeutic target.

Detection can be by any appropriate method, including for example, detecting the quantity of mRNA transcribed from the gene, or the quantity of cDNA produced from the reverse transcription of the mRNA transcribed from the gene, or the quantity of the polypeptide or protein encoded by the gene. These methods can be performed on a sample by sample basis or modified for high throughput analysis. Additionally, databases containing quantitative full or partial transcripts or protein sequences isolated from a cell sample can be searched and analyzed for the presence and amount of transcript or expressed gene product. The methods are particularly useful for aiding in the diagnosis of non-small cell lung cancer cell.

Another aspect of the invention is a screen to identify therapeutic agents that reverse or treat lung neoplasia and tumors, wherein the lung cell and/or tumor is characterized by the differential expression of at least one gene selected from EGFR-RS, RYK, TNFRSF25, TRPM7, KCP3, KIAA 1883 or UNC5H2. (See Table 1). The method comprises contacting the cell previously identified as possessing this genotype with an effective amount of a potential agent and assaying for reversal of the neoplastic condition.

TABLE 1

| | | | | | | RECEP- | TUMOR |
|---|---|---|---|---|---|---|---|
| | | | TRANS- | SIGNAL | DEATH | TOR/ | VERSUS |
| | LOCUS | SAGE TAG | MEMBRANE | PEPTIDE | DOMAIN | KINASE | NORMAL |
| GENE | LINK | SEQUENCE | REGION 1 | 1 | 1 | 1 | (LUNG)2 |

| | | | | | | | |
|---|---|---|---|---|---|---|---|
| EGFR-RS | 64285 | TGGCCAATAA | YES | NO | NO | YES | T > N |
| RYK | 6259 | GAAAACTGTT | YES | NO | NO | YES | T > N |
| TNFRSF25 | 8718 | GGGCTGGACG | YES | YES | YES | YES | T > N |
| (aka DR3) | | | | | | | |
| TRPM7 | 54822 | AATGCTGTTT | YES | NO | NO | YES | T > N |
| UNC5H2 | 219699 | GGTTTTAGTT | n/d | n/d | YES | YES | N > T |
| KCP3 | 200634 | TCTGCAGGGG | YES | YES | NO | YES | T > N |
| KIAA 1883 | 114783 | TGCCAAACGG | YES | YES | NO | YES | T > N |

1. Predicted functions based on sequence analysis.
2. Expression analysis - lung tumor versus normal lung cells.
n/d = not done
* = www.ncbi.nlm.nih.gov/LocusLink/list.cgi.

Further provided by this invention is a method for monitoring lung cancer in a subject by assaying, at different times, the expression level of at least one gene identified in Table 1 and comparing the expression level of the gene (transcript or expression product) to determine if expression has increased or decreased, thereby monitoring lung cancer in the subject. A kit for use in a diagnostic method or drug screen is further provided herein. The kit comprises at least one agent (e.g., probe, primer or antibody) that detects expression of at least one gene identified in Table 1 and instructions for use.

Further provided are polynucleotides encoding the proteins, fragments thereof, or polypeptides, (also referred to herein as a gene expression product), gene delivery vehicles comprising these polynucleotides and host cells comprising these polynucleotides. The proteins, polypeptides or fragments thereof are also useful to generate antibodies that specifically recognize and bind to these molecules. The antibodies can be polyclonal or monoclonal. These antibodies can be used to isolate protein or polypeptides expressed from the genes identified in Table 1. These antibodies are further useful for passive immunotherapy when administered to a subject.

The invention also provides isolated host cells and recombinant host cells that contain a gene of Table 1 or its expression product and/or fragments of either. The cells can be prokaryotic or eukaryotic and by way of example only, can be any one or more of bacterial, yeast, animal, mammalian, human, and particular subtypes thereof, e.g., stem cells, antigen presenting cells (APCs) such as dendritic cells (DCs) or T cells.

In addition, the invention provides methods for active immunotherapy, such as, inducing an immune response in a subject by delivering the proteins, polypeptides and fragments of either, as described herein, to the subject. In one aspect, the proteins and/or polypeptides can be delivered in the context of an MHC molecule.

The invention also provides immune effector cells raised in vivo or in vitro in the presence and at the expense of an antigen presenting cell that presents a polypeptide fragment expressed from a gene identified in Table 1, supra, in the context of an MHC molecule. The invention also provides a method of adoptive immunotherapy comprising administering an effective amount of these immune effector cells to a subject.

Yet another embodiment of the present invention is a method of reversing the neoplastic condition of a lung cell, wherein the cell is characterized by differential expression of a gene identified in Table 1, by contacting the cell with a therapeutic agent.

### BRIEF DESCRIPTION OF THE SEQUENCE LISTINGS

SEQ ID NO: 1 is a polynucleotide sequence encoding an EGFR-RS polypeptide.

SEQ ID NO:2 is a polypeptide sequence encoded from an EGFR-RS gene.

SEQ ID NO:3 is a polynucleotide sequence encoding a RYK polypeptide.

SEQ ID NO:4 is a polypeptide sequence encoded from an RYK gene.

SEQ ID NO:5 is a polynucleotide sequence encoding a TNFRSF25 polypeptide.

SEQ ID NO:6 is a polypeptide sequence encoded from a TNFRSF25 gene.

SEQ ID NO:7 is a polynucleotide sequence encoding a TRPM7 polypeptide.

SEQ ID NO:8 is a polypeptide sequence encoded from a TRPM7 gene.

SEQ ID NO:9 is a polynucleotide sequence encoding a UNC5H2 polypeptide.

SEQ ID NO:10 is a polypeptide sequence encoded from a UNC5H2 gene.

SEQ ID NO:11 is a polynucleotide sequence encoding a KCP3 polypeptide.

SEQ ID NO:12 is a polypeptide sequence encoded from a KCP3 gene.

SEQ ID NO:13 is a polynucleotide sequence encoding a KIAA1883 polypeptide.

SEQ ID NO:14 is a polypeptide sequence encoded from a KIAA 1883 gene.

### MODES FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

### DEFINITIONS

The practice of the present invention will employ, unless otherwise indicated, conventional techniques of immunology, molecular biology, microbiology, cell biology and recombinant DNA, which are within the skill of the art. See, e.g., Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

As used herein, certain terms have the following defined meanings.

As used in the specification and claims, the singular form “a”, “an” and “the” include plural references unless the context clearly dictates otherwise. For example, the term “a cell” includes a plurality of cells, including mixtures thereof.

The terms “polynucleotide” and “oligonucleotide” are used interchangeably, and refer to a polymeric form of nucleotides of any length, either deoxyribonucleotides or ribonucleotides, or analogs thereof. Polynucleotides can have any three-dimensional structure, and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide can comprise modified nucleotides, such as methylated nucleotides and nucleotide analogs. If present, modifications to the nucleotide structure can be imparted before or after assembly of the polymer. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for guanine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

A “gene” refers to a polynucleotide containing at least one open reading frame (ORF) that is capable of encoding a particular polypeptide or protein after being transcribed and translated. Any of the polynucleotides sequences described herein may be used to identify larger fragments or full-length coding sequences of the gene with which they are associated. Methods of isolating larger fragment sequences are known to those of skill in the art.

A “gene product” or alternatively a “gene expression product” refers to the amino acid (e.g., peptide or polypeptide) generated when a gene is transcribed and translated.

The term “polypeptide” is used interchangeably with the term “protein” and in its broadest sense refers to a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

“Under transcriptional control” is a term well understood in the art and indicates that transcription of a polynucleotide sequence, usually a DNA sequence, depends on its being operatively linked to an element which contributes to the initiation of, or promotes, transcription. “Operatively linked” refers to a juxtaposition wherein the elements are in an arrangement allowing them to function.

As used herein, the term “EGFR-RS gene” refers to at least the ORF of a contiguous polynucleotide sequence and that encodes a protein or polypeptide having the biological activity as set forth in Table 1. SEQ ID NO: 1 is one example of an EGFR-RS gene, and others are known in the art, examples of which include, but are not limited to the sequences set forth under GenBank Accession Nos: NM_{—}022450.2 and the sequences that encode EGFR-RS gene expression products as defined herein. Also included within this definition are biologically equivalent sequences such as those sequences that code for the polypeptide of SEQ ID NO:2 and those having at least 90% or alternatively, at least 95% sequence homology to an exemplary sequence, such as SEQ ID NO: 1, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art. See for example, UniGene Cluster Hs.57988. Polynucleotide fragments are also known in the art, and include but are not limited to GenBank Accession Nos.: AA653398.1; AI671048.1; AA858091.1 and AI128203.1. These are particularly useful as probes or primers.

As used herein, the term “EGFR-RS gene expression product, protein or polypeptide” includes the amino acid sequence of SEQ ID NO: 2 as well as the amino acid sequences transcribed and translated from the EGFR-RS genes identified above, without regard to the gene expression system, e.g., bacterial or other prokaryotic cell, yeast cell, or mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO:2 and which have the biological activity as shown in Table 1. Examples of homologous amino acid sequences include, but are not limited to polypeptides having the amino acid sequence of SEQ ID NO: 2 or another EGFR-RS gene expression product that has been modified by conservative amino acid substitutions.

As used herein, the term “RYK gene” refers to at least the ORF of a contiguous polynucleotide sequence that encodes a protein or polypeptide having the biological activity as set forth in Table 1. Sequence ID NO: 3 is one example of an RYK gene, and others are known in the art, examples of which include, but are not limited to the sequences set forth under NCBI RefSeq NM 002958.1 and GenBank Accession Nos.: NB 00_{—}2958.1, X96588.1, BC0217001, S59184.1; X69970.1; X96588.1, and the sequences that encode RYK gene expression products as defined herein. Also included within this definition are biologically equivalent sequences such as those sequences that code for the polypeptide of SEQ ID NO:4 and those having at least 90% or alternatively, at least 95% sequence homology to an exemplary sequence, such as SEQ ID NO: 3, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art. See for example, UniGene Cluster Hs.79350. Polynucleotide fragments are also known in the art, and include but are not limited to GenBank Accession Nos.: AA845370.1; AI698284.1; and AI500529.1. These are particularly useful as probes or primers.

As used herein, the term “RYK gene expression product, protein or polypeptide” includes the amino acid sequence of SEQ ID NO: 4 as well as the amino acid sequences transcribed and translated from the RYK genes identified above, without regard to the gene expression system, e.g., bacterial or other prokaryotic cell, yeast cell, mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO: 4 and which have the biological activity as shown in Table 1. Examples of homologous amino acid sequences include, but are not limited to polypeptides have the amino acid sequence of SEQ ID NO: 4 or other RYK gene expression product that has been modified by conservative amino acid substitutions.

As used herein, the term “TNFRSF25” or “DR3” gene” refers to at least the ORF of a contiguous polynucleotide sequence that encodes a protein or polypeptide having the biological activity as set forth in Table 1. Chinnaiyan A. M. et al. (1996) Science 274(5289):990-992. Sequence ID NO: 5 is one example of a DR3 gene, and others are known in the art, examples of which include, but are not limited to the sequences set forth under GenBank Accession Nos.: NM_{—}003790.2; NM_{—}148974.1; NM_{—}148973.1; NM_{—}148972.1, NM_{—}148971.1, and the sequences that encode TNFRSF25 gene expression products as defined herein. Also included within this definition are biologically equivalent sequences such as those sequences that code for the polypeptide of SEQ ID NO: 6 and those having at least 90% or alternatively, at least 95% sequence homology to an exemplary sequence, such as SEQ ID NO: 5, and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art. See for example, UniGene Cluster Hs.180338. Polynucleotide fragments are also known in the art, and include but are not limited to GenBank Accession Nos.: AI203624.1; AI424936.1; AI140043.1; and AI700459.1. These are particularly useful as probes or primers.

As used herein, the term “TNFRSF25 Gene expression product, protein or polypeptide” includes the amino acid sequence of SEQ ID NO: 6 as well as the amino acid sequences transcribed and translated from the TNFRSF25 genes identified above, without regard to the gene expression system, e.g., bacterial or other prokaryotic cell, yeast cell, or mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO: 6 and which have the biological activity as shown in Table 1. Examples of homologous amino acid sequences include, but are not limited to polypeptides having the amino acid sequence of SEQ ID NO: 6 or another TNFRSF25 gene expression product that has been modified by conservative amino acid substitutions.

As used herein, the term “TRPM7 gene” refers to at least the ORF of a contiguous polynucleotide sequence that encodes a protein or polypeptide having the biological activity as set forth in Table 1. Sequence ID NO: 7 is one example of a TRPM7 gene, and others are known in the art examples of which include, but are not limited to the sequences set forth under GenBank Accession No. NM017672.1 and the sequences that encode TRPM7 gene expression products as defined herein. Also included within this definition are biologically equivalent sequences such as those sequences that code for the polypeptide of SEQ ID NO: 8 and those having at least 90% or alternatively, at least 95% sequence homology to an exemplary sequence, such as SEQ ID NO: 7 and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art. See for example, UniGene Cluster Hs.267914. Polynucleotide fragments are also known in the art, and include but are not limited to GenBank Accession Nos.: AI687022.1; AI926826.1; AI761540.1; and AI814269.1. These are particularly useful as probes or primers.

As used herein, the term “TRPM7 gene expression product, protein or polypeptide” includes the amino acid sequence of SEQ ID NO: 8 as well as the amino acid sequences transcribed and translated from the TRPM7 genes identified above, without regard to the gene expression system, e.g., bacterial or other prokaryotic cell, yeast cell, or mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO: 8 and which have the biological activity as shown in Table 1. Examples of homologous amino acid sequences include, but are not limited to polypeptides having the amino acid sequence of SEQ ID NO: 8 or another TRPM7 gene expression product that has been modified by conservative amino acid substitutions.

As used herein, the term “UNC5H2 gene” refers to at least the ORF of a contiguous polynucleotide sequence that encodes a protein or polypeptide having the biological activity as set forth in Table 1. Sequence ID NO: 9 is one example of an UNC5H2 gene, and others are known in the art, examples of which include, but are not limited to the sequences set forth under GenBank Accession No. NM_{—}170744.1 and the sequences that encode UNC5H2 gene expression products as defined herein. Also included within this definition are biologically equivalent sequences such as those sequences that code for the polypeptide of SEQ ID NO: 10 and those having at least 90% or alternatively, at least 95% sequence homology to an exemplary sequence, such as SEQ ID NO: 9 and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art. See for example, UniGene Cluster Hs.183918. Polynucleotide fragments are also known in the art, and include but are not limited to GenBank Accession Nos.: AK022859.1; AK094595.1; and AY126437.1. These are particularly useful as probes or primers.

As used herein, the term “UNC5H2” gene expression product, protein or polypeptide” includes the amino acid sequence of SEQ ID NO: 10 as well as the amino acid sequences transcribed and translated from the UNC5H2 genes identified above, without regard to the gene expression system, e.g., bacterial or other prokaryotic cell, yeast cell, or mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO: 10 and which have the biological activity as shown in Table 1. Examples of homologous amino acid sequences include, but are not limited to polypeptides having the amino acid sequence of SEQ ID NO: 10 or another UNC5H2 gene expression product that has been modified by conservative amino acid substitutions.

As used herein, the term “KCP3 gene” refers to at least the ORF of a contiguous polynucleotide sequence that encodes a protein or polypeptide having the biological activity as set forth in Table 1. SEQ ID NO: 11 is one example of an KCP3 gene, and others are known in the art examples of which include, but are not limited to the sequences set forth under GenBank Accession No. NM_{—}173853 and the sequences that encode KCP3 gene expression products as defined herein. Also included within this definition are biologically equivalent sequences such as those sequences that code for the polypeptide of SEQ ID NO: 12 and those having at least 90% or alternatively, at least 95% sequence homology to an exemplary sequence, such as SEQ ID NO: 11 and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art. Polynucleotide fragments are also known in the art, and include but are not limited to GenBank Accession Nos.: AA531276.1; AI831051.1; and W93943.1. These are particularly useful as probes or primers.

As used herein, the term “KCP3” gene expression product, protein or polypeptide” includes the amino acid sequence of SEQ ID NO: 12 as well as the amino acid sequences transcribed and translated from the KCP3 genes identified above, without regard to the gene expression system, e.g., bacterial or other prokaryotic cell, yeast cell, or mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO: 12 and which have the biological activity as shown in Table 1. Examples of homologous amino acid sequences include, but are not limited to polypeptides having the amino acid sequence of SEQ ID NO: 12 or another KCP3 gene expression product that has been modified by conservative amino acid substitutions.

As used herein, the term “KIAA1883 gene” refers to at least the ORF of a contiguous polynucleotide sequence that encodes a protein or polypeptide having the biological activity as set forth in Table 1. Sequence ID NO: 13 is one example of an KIAA1883 gene, and others are known in the art, examples of which include, but are not limited to the sequences set forth under GenBank Accession No. XM_{—}055866 and AB067470, and the sequences that encode TRPM7 gene expression products as defined herein. Also included within this definition are biologically equivalent sequences such as those sequences that code for the polypeptide of SEQ ID NO: 14 (or alternatively, the sequence identified under GenBank Accession Nos.: BAB67776) and those having at least 90% or alternatively, at least 95% sequence homology to an exemplary sequence, such as SEQ ID NO: 13 and as determined by percent identity sequence analysis run under default parameters. Also within this definition are biologically equivalent genes or polynucleotides that are identified by the ability to hybridize under conditions of high stringency to the minus strand. It may be desirable to use non-human genes, the polynucleotide sequences of which are known in the art. See for example, UniGene Cluster Hs.281328. Polynucleotide fragments are also known in the art, and include but are not limited to GenBank Accession Nos.: AW044638.1; H51141.1; AW015335.1; and BE466321.1. These are particularly useful as probes or primers.

As used herein, the term “KIA1883 gene expression product, protein or polypeptide” includes the amino acid sequence of SEQ ID NO: 14 as well as the amino acid sequences transcribed and translated from the KIA1883 genes identified above, without regard to the gene expression system, e.g., bacterial or other prokaryotic cell, yeast cell, or mammalian cell such as a simian, bovine or human cell. The term includes isolated, naturally occurring polypeptides isolated from tissue samples as well as recombinantly produced proteins and polypeptides. The term also includes polypeptides having the amino acid sequences that are at least 90% or alternatively at least 95% homologous to SEQ ID NO: 14 and which have the biological activity as shown in Table 1. Examples of homologous amino acid sequences include, but are not limited to polypeptides having the amino acid sequence of SEQ ID NO: 14 or another KIA1883 gene expression product that has been modified by conservative amino acid substitutions.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The term “isolated” means separated from constituents, cellular and otherwise, in which the polynucleotide, peptide, polypeptide, protein, antibody, or fragments thereof, are normally associated with in nature. In one aspect of this invention, an isolated polynucleotide is separated from the 3′ and 5′ contiguous nucleotides with which it is normally associated with in its native or natural environment, e.g., on the chromosome. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known in the art to be capable of mediating transfer of genes to mammalian cells.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; recombinant yeast cells, metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a therapeutic gene. As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., WO 95/27071. Ads are easy to grow and do not require integration into the host cell genome. Recombinant Ad derived vectors, particularly those that reduce the potential for recombination and generation of wild-type virus, have also been constructed. See, WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include several non-viral vectors, including DNA/liposome complexes, recombinant yeast cells, and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., TCR, CD3 or CD4.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “primer” is a short polynucleotide, generally with a free 3′—OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in “PCR: A PRACTICAL APPROACH” (M. MacPherson et al., IRL Press at Oxford University Press (1991)). All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra.

An expression “database” denotes a set of stored data that represent a collection of sequences, which in turn represent a collection of biological reference materials.

The term “cDNAs” refers to complementary DNA that are mRNA molecules present in a cell or organism made into cDNA with an enzyme such as reverse transcriptase. A “cDNA library” is a collection of all of the mRNA molecules present in a cell or organism, all turned into cDNA molecules with the enzyme reverse transcriptase or an equivalent, then inserted into “vectors” (other DNA molecules that can continue to replicate after addition of foreign DNA). Exemplary vectors for libraries include bacteriophage (also known as “phage”), viruses that infect bacteria, for example, lambda phage. The library can then be probed for the specific cDNA (and thus mRNA) of interest.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. “Differentially expressed” as applied to a gene, refers to the differential production of the mRNA transcribed and/or translated from the gene or the protein product encoded by the gene. A differentially expressed gene may be overexpressed or underexpressed as compared to the expression level of a normal or control cell. In one aspect, it refers to a differential that is 2.5 times, preferably 5 times, or preferably 10 times higher or lower than the expression level detected in a control sample. The term “differentially expressed” also refers to nucleotide sequences in a cell or tissue which are expressed where silent in a control cell or not expressed where expressed in a control cell.

As used herein, “solid phase support” or “solid support”, used interchangeably, is not limited to a specific type of support. Rather a large number of supports are available and are known to one of ordinary skill in the art. Solid phase supports include silica gels, resins, derivatized plastic films, glass beads, cotton, plastic beads, alumina gels, microarrays and chips. As used herein, “solid support” also includes synthetic antigen-presenting matrices, cells, and liposomes. A suitable solid phase support may be selected on the basis of desired end use and suitability for various protocols. For example, for peptide synthesis, solid phase support may refer to resins such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TentaGel®, Rapp Polymere, Tubingen, Germany) or polydimethylacrylamide resin (obtained from Milligen/Biosearch, California).

A polynucleotide also can be attached to a solid support for use in high throughput screening assays. PCT WO 97/10365, for example, discloses the construction of high density oligonucleotide chips. See also, U.S. Pat. Nos. 5,405,783; 5,412,087; and 5,445,934. Using this method, the probes are synthesized on a derivatized glass surface also known as chip arrays. Photoprotected nucleoside phosphoramidites are coupled to the glass surface, selectively deprotected by photolysis through a photolithographic mask, and reacted with a second protected nucleoside phosphoramidite. The coupling/deprotection process is repeated until the desired probe is complete.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

A polynucleotide or polynucleotide region (or a polypeptide or polypeptide region) has a certain percentage (for example, 80%, 85%, 90%, or 95%) of “sequence identity” to another sequence means that, when aligned, that percentage of bases (or amino acids) are the same in comparing the two sequences. This alignment and the percent homology or sequence identity can be determined using software programs known in the art, for example those described in CURRENT PROTOCOLS IN MOLECULAR BIOLOGY (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/cgi-bin/BLAST.

Hyperplasia is a form of controlled cell proliferation involving an increase in cell number in a tissue or organ, without significant alteration in structure or function. Metaplasia is a form of controlled cell growth in which one type of fully differentiated cell substitutes for another type of differentiated cell. Metaplasia can occur in epithelial or connective tissue cells. Atypical metaplasia involves a somewhat disorderly metaplastic epithelium.

As used herein, the terms “neoplastic cells”, “neoplasia”, “tumor”, “tumor cells”, “cancer” and “cancer cells”, (used interchangeably) refer to cells which exhibit relatively autonomous growth, so that they exhibit an aberrant growth phenotype characterized by a significant loss of control of cell proliferation (i.e., de-regulated cell division). Neoplastic cells can be malignant or benign. A metastatic cell or tissue means that the cell can invade and destroy neighboring body structures.

“Suppressing” tumor growth indicates a growth state that is curtailed when compared to growth without contact with educated, antigen-specific immune effector cells described herein. Tumor cell growth can be assessed by any means known in the art, including, but not limited to, measuring tumor size, determining whether tumor cells are proliferating using a ³H-thymidine incorporation assay, or counting tumor cells. “Suppressing” tumor cell growth means any or all of the following states: slowing, delaying, and stopping tumor growth, as well as tumor shrinkage.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975)).

An “effective amount” is an amount sufficient to effect beneficial or desired results such as prevention or treatment. An effective amount can be administered in one or more administrations, applications or dosages.

A “subject,” “individual” or “patient” is used interchangeably herein, which refers to a vertebrate, preferably a mammal, more preferably a human. Mammals include, but are not limited to, murines, simians, humans, farm animals, sport animals, and pets.

A “control” is an alternative subject or sample used in an experiment for comparison purpose. A control can be “positive” or “negative”. For example, where the purpose of the experiment is to determine a correlation of an altered expression level of a gene with a particular type of cancer, it is generally preferable to use a positive control (a subject or a sample from a subject, carrying such alteration and exhibiting symptoms characteristic of that disease), and a negative control (a subject or a sample from a subject lacking the altered expression and clinical syndrome of that disease).

### DIAGNOSTIC METHODS

As noted above, this invention provides various methods for aiding in the diagnosis of the neoplastic state of a lung cell that is characterized by abnormal cell growth in the form of, e.g., malignancy, hyperplasia or metaplasia. The methods are particularly useful for aiding in the diagnosis of non-small cell lung cancer cell. The neoplastic state of a cell generally is determined by noting whether or not the growth of the cell is governed by the usual limitation of normal growth. For the purposes of this invention, the term also is to include genotypic changes that occur prior to detection of this growth in the form of a tumor and are causative of these phenotypic changes. The phenotypic changes associated with the neoplastic state of a cell (a set of in vitro characteristics associated with a tumorigenic ability in vivo) include a more rounded cell morphology, looser substratum attachment, loss of contact inhibition, loss of anchorage dependence, release of proteases such as plasminogen activator, increased sugar transport, decreased serum requirement, expression of fetal antigens and the like. (See, Luria et al. (1978) GENERAL VIROLOGY, 3^{d} edition, 436-446 (John Wiley & Sons, New York)).

Accordingly, one embodiment is a method of diagnosing the condition of a lung cell by screening for the presence of a differentially expressed gene isolated from a sample containing or suspected of containing a lung cell, in which the differential expression of the gene is indicative of the neoplastic state of the lung cell. In one aspect, the gene is expressed more in a neoplastic lung cell or a lung tumor cell as compared to normal lung cell, and is selected from EGFR-RS, RYK, TNFRSF25, TRPM7, KCP3, and KIAA 1883. In another aspect, the gene is expressed more in a normal lung cell as compared to a neoplastic lung cell, e.g., UNC5H2. Detection can be by any appropriate method, including for example, detecting the quantity of mRNA transcribed from the gene, or the quantity of cDNA produced from the reverse transcription of the mRNA transcribed from the gene, or the quantity of the polypeptide or protein encoded by the gene. Probes for each of these methods are provided in Table 1. These methods can be performed on a sample by sample basis or modified for high throughput analysis. Additionally, databases containing quantitative full or partial transcripts or protein sequences isolated from a cell sample can be searched and analyzed for the presence and amount of transcript or expressed gene product. In one aspect, the database contains at least one of the sequences shown in Table 1.

Epidermal Growth Factor Receptor-Related Sequence (referred to as “EGFR-RS” in Table 1) is a member of the epidermal growth factor receptor (EGFR) family which in turn is a member of a larger family of closely related transmembrane receptors (erbB receptors), all of which appear to be involved in the regulation of cellular growth, replication and/or differentiation. ErbB includes four receptors, including EGFR (erbB-1), Her 2 (erbB-2), Her 3 (erbB-3) and Her 4 (erbB-4). All four receptors share similar structure including an extracellular region which consists of glycosylated domains and binds to extracellular ligand, a short helical transmembrane domain secured by a single hydrophobic sequence and an intracellular tyrosine kinase domain that is responsible for initiating and regulating intracellular signaling. One known exception to this is the erbB-3 receptor, which is activated through other erbB tyrosine kinases, as erbB-3 lacks its own kinase activity.

The RYK gene encodes a protein comprised of two polypeptides, an approximately 80 kD polypeptide and an approximately 45 kD polypeptide. The gene is overexpressed in neuroblastomas, Wilms tumor, melanoma, ovarian cancer (epithelium and blood) and in leukemic cells. Katso et al. (2000) Clin. Cancer Res. 6(8):3271-3281 has reported that overexpression of RYK is a prognostic indicator for ovarian cancer. Overexpression of the gene has been reported to be transforming in vitro and in vivo. Katso et al. (1999) Cancer Res. 59:2265-2270. RYK knockout mice have also been reported to be small and die within one week due to cleft palate.

Excessive activation of EGFR occurring through receptor mutation, increased concentration of ligand (extracellular or intracellular), increased receptor number and/or decreased receptor turnover can drive the initiation of uncontrolled cellular growth. Wells A. (1993) Inter. J. of Bio. and Cell Bio. 31:637-643. Mutations in any domain of EGFR or components of its pathways may lead to overactive signaling, which may ultimately result in the development and/or metastasis of cancer.

EGFR has demonstrated high affinity to the ligands including, but not necessarily limited to: epidermal growth factor (EGF), transforming growth factor-alpha, amphiregulin, heparin-binding EGF, betacellulin and epiregulin. Fedi P. et al. (2000), pages 33 to 55 in CANCER MEDICINE, 5^{th} Ed. Bast et al. eds, B. C. Decker Inc. Once the extracellular domain binds to ligand, the bound receptor forms either a homodimer or heterodimer with a neighboring erbB receptor. Dimerization initiates intramolecular phosphorylation of the EGFR tyrosine kinase and substrates, which generates a downstream cascade of catalytic events that ultimately carries growth signal into the nucleus of the cell. Following activation of EGFR, the receptor is internalized through endocytosis and either degraded through lysosomal processes or recycled to the cell surface.

Applicants have discovered overexpression of this gene in solid tumors of epithelium origin, e.g., lung carcinomas, breast carcinomas, metastatic breast carcinomas, primary ductal-breast carcinoma, mammary gland breast carcinoma and pleural effusion mammary gland carcinoma.

Receptor-Like Tyrosine Kinase (referred to as “RYK” in Table 1) was reported by Hovens et al. (1992) PNAS 89:11818-11822, to be a novel member of the family of growth factor receptor protein tyrosine kinases. Comparison of mouse and human RYK cDNA sequences demonstrated a very high degree of sequence identity (Stacker et al. (1993) Oncogene 8:1347-1356). By genetic linkage analysis with recombinant inbred strains of mice, Gough et al. (1995) Mammalian Gen. 6:255-256, identified 2 distinct mouse Ryk loci (Ryk1 and Ryk2) and showed that they mapped to chromosomes 9 and 12, respectively. A similar arrangement of RYK-related loci had been determined in the human: in situ hybridization placed human RYK1 on 3q22 and RYK2 on 17q. Although previously reported to be an expression marker for ovarian cancers (Wang et al. (1996) Mol. Med. 2(2):189-203) Applicants are the first to report expression on lung cancer cells.

Tumor Necrosis Factor Receptor Superfamily, Member 25 (“TNFRSF25”) is also known as Death Receptor 3 (“DR3”), is a member of the mammalian tumor necrosis factor receptor (TNFR) family. The TNFR family are cell-surface proteins that interact with a corresponding TNF-related ligand family. The receptors share homology in the extracellular domain, which contains 3 to 6 cysteine-rich pseudorepeats, but are generally not related in their cytoplasmic regions. However, the intracellular domains (ICDs) of TNFR family members, e.g., TNFR1 and FAS/APO1/CD95, can activate apoptotic cell death. They have a region of homology in an oligomerization interface known as the death domain.

This gene was found to be overexpressed in breast carcinoma cells and induced rapid apoptosis in 293 cells. The gene also induced NF-kB (unlike Fas) when 293 cells were cotransfected with DR3 and a NF-kB luciferase plasmid. The gene is reported to be expressed in peripheral blood lymphocytes (PBLs) thymus, spleen, colon and small intestine. Kitson et al. (1996) Nature 384:372-375 previously reported that no expression has been reported in ovaries, testis, prostate, pancreas, kidney, skeletal muscle, liver lung, placenta, brain and heart.

Transient Receptor Potential Cation Channel, Subfamily M, Member 7 (referred to as “TRPM7” in Table 1) is a mammalian homolog of the Drosophila transient receptor potential (trp) protein, which are known ion channels thought to mediate capacitative calcium entry into the cell. As a channel, these receptors conduct calcium and monovalent cations to depolarize cells and increase intracellular calcium. As a kinase, it is capable of phosphorylating itself and other substrates. The kinase activity is necessary for channel function, as shown by its dependence on intracellular ATP and by the kinase mutants.

Transmembrane Receptor Unc5H2 (referred to as “UNC5H2” in Table 1) was reported by Komatsuzaki K. et al. (2002) Biochem. Biophys. Res. Comm. 297(4):898-905, to be a G protein which are known to regulate a number of cellular functions including cell migration, proliferation, and differentiation. It is also reported that UNC5H2 is widely expressed particularly in cells which migrate.

KIAA1883 is one of 60 cDNA clones identified as an extension of a sequencing project to identify large proteins of unidentified genes. Nagase et al. (2001) DNA Res. 8:179-187. Nagase et al. reported that the cDNAs were isolated from libraries derived from human fetal brain, adult whole brain and amygdala.

For the purpose of illustration only, gene expression is determined by noting the amount (if any, e.g., altered) expression of the gene in the test system at the level of an mRNA transcribed from at least one gene identified in Table 1. In a separate embodiment, augmentation of the level of the polypeptide or protein encoded by the gene is indicative of the presence of the neoplastic condition of the cell. In yet a further embodiment, a decrease in the level of polypeptide or protein encoded by the gene is indicative of the neoplastic condition. The method can be used for aiding in the diagnosis of lung cancer such as non-small cell lung cancer by detecting a genotype that is correlated with a phenotype characteristic of primary lung tumor cells. Thus, by detecting this genotype prior to tumor growth, one can predict a predisposition to cancer and/or provide early diagnosis and treatment.

Cell or tissue samples used for this invention encompass body fluid, solid tissue samples, tissue cultures or cells derived there from and the progeny thereof, and sections or smears prepared from any of these sources, or any other samples that may contain a lung cell having a gene described herein. In one embodiment, the sample comprises cells prepared from a subject's lung tissue.

In assaying for an alteration in mRNA level, nucleic acid contained in the aforementioned samples is first extracted according to standard methods in the art. For instance, mRNA can be isolated using various lytic enzymes or chemical solutions according to the procedures set forth in Sambrook et al. (1989) supra, or extracted by nucleic-acid-binding resins following the accompanying instructions provided by manufactures. The mRNA of a proto-oncogene of interest contained in the extracted nucleic acid sample is then detected by hybridization (e.g., Northern blot analysis) and/or amplification procedures according to methods widely known in the art or based on the methods exemplified herein.

Nucleic acid molecules having at least 10 nucleotides and exhibiting sequence complementarity or homology to at least one gene identified in Table 1 find utility as hybridization probes. It is known in the art that a “perfectly matched” probe is not needed for a specific hybridization. Minor changes in probe sequence achieved by substitution, deletion or insertion of a small number of bases do not affect the hybridization specificity. In general, as much as 20% base-pair mismatch (when optimally aligned) can be tolerated. Preferably, a probe useful for detecting mRNA is at least about 80% identical to the homologous region of comparable size contained in the genes or polynucleotides identified in Table 1 identified sequences, which have the Locus Link numbers identified in Table 1. In one aspect, the probe is 85% identical to the corresponding gene sequence after alignment of the homologous region, or alternatively, it exhibits 90% identity. Additional probes can be derived from sequences for the genes identified by the Locus Link Nos. provided in Table 1, or to a homologous region of comparable size contained in the previously identified sequences, which have the Locus Link Nos. identified in Table 1. These probes can be used in radioassays (e.g., Southern and Northern blot analysis) to detect, prognose, diagnose or monitor various neoplastic states resulting from differential expression of a gene of interest. The total size of fragment, as well as the size of the complementary stretches, will depend on the intended use or application of the particular nucleic acid segment. Smaller fragments derived from the known sequences will generally find use in hybridization embodiments, wherein the length of the complementary region may be varied, such as between about 10 and about 100 nucleotides, or even full length according to the complementary sequences one wishes to detect.

In one aspect, nucleotide probes having complementary sequences over stretches greater than about 10 nucleotides in length are used, so as to increase stability and selectivity of the hybrid, and thereby improving the specificity of particular hybrid molecules obtained. Alternatively, one can design nucleic acid molecules having gene-complementary stretches of more than about 25 or alternatively more than about 50 nucleotides in length, or even longer where desired. Such fragments may be readily prepared by, for example, directly synthesizing the fragment by chemical means, by application of nucleic acid reproduction technology, such as the PCR™ technology with two priming oligonucleotides as described in U.S. Pat. No. 4,603,102 or by introducing selected sequences into recombinant vectors for recombinant production. In one aspect, a probe is about 50 to about 75, nucleotides or alternatively, about 50 to about 100 nucleotides in length.

In certain embodiments, it will be advantageous to employ nucleic acid sequences as described herein in combination with an appropriate means, such as a label, for detecting hybridization and therefore complementary sequences. A wide variety of appropriate indicator means are known in the art, including fluorescent, radioactive, enzymatic or other ligands, such as avidin/biotin, which are capable of giving a detectable signal. One can employ a fluorescent label or an enzyme tag, such as urease, alkaline phosphatase or peroxidase, instead of radioactive or other environmental undesirable reagents. In the case of enzyme tags, colorimetric indicator substrates are known which can be employed to provide a means visible to the human eye or spectrophotometrically, to identify specific hybridization with complementary nucleic acid-containing samples.

Hybridization reactions can be performed under conditions of different “stringency”. Relevant conditions include temperature, ionic strength, time of incubation, the presence of additional solutes in the reaction mixture such as formamide, and the washing procedure. Higher stringency conditions are those conditions, such as higher temperature and lower sodium ion concentration, which require higher minimum complementarity between hybridizing elements for a stable hybridization complex to form. Conditions that increase the stringency of a hybridization reaction are widely known and published in the art. See, for example, Sambrook et al. (1989) supra.

The nucleotide probes of the present invention can also be used as primers and detection of genes or gene transcripts that are differentially expressed in certain body tissues. Additionally, a primer useful for detecting the aforementioned differentially expressed mRNA is at least about 80% identical to the homologous region of comparable size contained in the previously identified sequences, which have the Locus Link Nos. numbers identified in Table 1. For the purpose of this invention, amplification means any method employing a primer-dependent polymerase capable of replicating a target sequence with reasonable fidelity. Amplification may be carried out by natural or recombinant DNA-polymerases such as T7 DNA polymerase, Klenow fragment of E. coli DNA polymerase, and reverse transcriptase.

A known amplification method is PCR, MacPherson et al., PCR: A PRACTICAL APPROACH, (IRL Press at Oxford University Press (1991)). However, PCR conditions used for each application reaction are empirically determined. A number of parameters influence the success of a reaction. Among them are annealing temperature and time, extension time, Mg²⁺ ATP concentration, pH, and the relative concentration of primers, templates, and deoxyribonucleotides.

After amplification, the resulting DNA fragments can be detected by agarose gel electrophoresis followed by visualization with ethidium bromide staining and ultraviolet illumination. A specific amplification of differentially expressed genes of interest can be verified by demonstrating that the amplified DNA fragment has the predicted size, exhibits the predicated restriction digestion pattern, and/or hybridizes to the correct cloned DNA sequence.

The probes also can be attached to a solid support for use in high throughput screening assays using methods known in the art. PCT WO 97/10365 and U.S. Pat. Nos. 5,405,783; 5,412,087 and 5,445,934; for example, disclose the construction of high density oligonucleotide chips which can contain one or more of the sequences disclosed herein. Using the methods disclosed in U.S. Pat. Nos. 5,405,783; 5,412,087 and 5,445,934; the probes of this invention are synthesized on a derivatized glass surface. Photoprotected nucleoside phosphoramidites are coupled to the glass surface, selectively deprotected by photolysis through a photolithographic mask, and reacted with a second protected nucleoside phosphoramidite. The coupling/deprotection process is repeated until the desired probe is complete.

The expression level of a gene can also be determined through exposure of a nucleic acid sample to a probe-modified chip. Extracted nucleic acid is labeled, for example, with a fluorescent tag, preferably during an amplification step. Hybridization of the labeled sample is performed at an appropriate stringency level. The degree of probe-nucleic acid hybridization is quantitatively measured using a detection device, such as a confocal microscope. See U.S. Pat Nos. 5,578,832 and 5,631,734. The obtained measurement is directly correlated with gene expression level.

The probes and high density oligonucleotide probe arrays also provide an effective means of monitoring expression of the genes identified in Table 1. They are also useful to screen for compositions that upregulate or downregulate the expression of the genes identified in Table 1.

In another embodiment, the methods of this invention are used to monitor expression of the genes identified in Table 1 which specifically hybridize to the probes of this invention in response to defined stimuli, such as an exposure of a cell or subject to a drug.

In one embodiment, the hybridized nucleic acids are detected by detecting one or more labels attached to the sample nucleic acids. The labels may be incorporated by any of a number of means known to those of skill in the art. However, in one aspect, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g., fluorescein-labeled UTP and/or CTP) incorporates a label in to the transcribed nucleic acids.

Alternatively, a label may be added directly to the original nucleic acid sample (e.g., mRNA, polyA, mRNA, cDNA, etc.) or to the amplification product after the amplification is completed. Means of attaching labels to nucleic acids are known to those of skill in the art and include, for example nick translation or end-labeling (e.g., with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, Texas red, rhodamine, green fluorescent protein, and the like), radiolabels (e.g., ³H, ¹²⁵I, ³⁵S, ¹⁴C, or ³²P) enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

As described in more detail in WO 97/10365, the label may be added to the target (sample) nucleic acid(s) prior to, or after the hybridization. These are detectable labels that are directly attached to or incorporated into the target (sample) nucleic acid prior to hybridization. In contrast, “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see LABORATORY TECHNIQUES IN BIOCHEMISTRY AND MOLECULAR BIOLOGY, Vol. 24: Hybridization with Nucleic Acid Probes, P. Tijssen, ed. Elsevier, N.Y. (1993).

The nucleic acid sample also may be modified prior to hybridization to the high density probe array in order to reduce sample complexity thereby decreasing background signal and improving sensitivity of the measurement using methods known in the art, e.g., the methods disclosed in WO 97/10365.

Results from the chip assay are typically analyzed using a computer software program. See, for example, EP 0717 113 A2 and WO 95/20681. The hybridization data is read into the program, which calculates the expression level of the targeted gene(s) i.e., the genes identified in Table 1. This figure is compared against existing data sets of gene expression levels for diseased and healthy individuals. A correlation between the obtained data and that of a set of diseased individuals indicates the onset of a disease in the subject patient.

Also within the scope of this application is a data base useful for the detection of neoplastic lung tissue comprising one or more of the sequences (or parts thereof) of the genes listed Table 1.

These polynucleotide sequences are stored in a digital storage medium such that a data processing system for standardized representation of the genes that identify a lung cancer cell is compiled. The data processing system is useful to analyze gene expression between two cells by first selecting a cell suspected of being of a neoplastic phenotype or genotype and then isolating polynucleotides from the cell. The isolated polynucleotides are then sequenced. The sequences from the sample are compared with the sequence(s) present in the database using homology search techniques described above. In one aspect, greater than 90% is selected, or alternatively greater than 95% is selected, or alternatively greater than or equal to 97% sequence identity is selected, between the test sequence and at least one sequence identified in Table 1 or its complement, is a positive indication that the polynucleotide has been isolated from a lung cancer cell as defined above.

Alternatively, one can compare a sample against a database. Briefly, multiple RNAs are isolated from cell or tissue samples using methods known in the art and described for example, in Sambrook et al. (1989) supra. Optionally, the gene transcripts can be converted to cDNA. A sampling of the gene transcripts are subjected to sequence-specific analysis and quantified. These gene transcript sequence abundances are compared against reference database sequence abundances including normal data sets for diseased and healthy patients. The patient has the disease(s) with which the patient's data set most closely correlates which includes the overexpression of the transcripts identified herein.

Differential expression of the genes of interest can also be determined by examining the protein product. A variety of techniques are available in the art for protein analysis. They include but are not limited to radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and PAGE-SDS. One means to determine protein level involves (a) providing a biological sample containing polypeptides; and (b) measuring the amount of any immunospecific binding that occurs between an antibody reactive to the expression product of a gene of interest and a component in the sample, in which the amount of immunospecific binding indicates the level of the expressed proteins.

Antibodies that specifically recognize and bind to the protein products of these genes are required for these immunoassays. These may be purchased from commercial vendors or generated and screened using methods well known in the art. See Harlow and Lane (1988) supra. and Sambrook et al. (1989) supra. Alternatively, polyclonal or monoclonal antibodies that specifically recognize and bind the protein product of a gene of interest can be made and isolated using known methods.

In diagnosing malignancy, hyperplasia or metaplasia characterized by a differential expression of genes, one typically conducts a comparative analysis of the subject and appropriate controls. Preferably, a diagnostic test includes a control sample derived from a subject (hereinafter “positive control”), that exhibits the predicted change in expression of a gene of interest, e.g., at a level of at least 2.5 fold and clinical characteristics of the malignancy or metaplasia of interest. Alternatively, a diagnosis also includes a control sample derived from a subject (hereinafter “negative control”), that lacks the clinical characteristics of the neoplastic state and whose expression level of the gene at question is within a normal range. A positive correlation between the subject and the positive control with respect to the identified alterations indicates the presence of or a predisposition to said disease. A lack of correlation between the subject and the negative control confirms the diagnosis. In a preferred embodiment, the method is used for diagnosing lung cancer, preferably non-small lung cancer, on the basis of a differential expression of a gene of Table 1

There are various methods available in the art for quantifying mRNA or protein level from a cell sample and indeed, any method that can quantify these levels is encompassed by this invention. For example, determination of the mRNA level of the aforementioned genes may involve, in one aspect, measuring the amount of mRNA in a sample isolated from the lung cell by hybridization or quantitative amplification using at least one oligonucleotide probe that is complementary to the mRNA. Determination of the aforementioned gene products requires measuring the amount of immunospecific binding that occurs between an antibody reactive to the gene product of a gene identified in Table 1. To detect and quantify the immunospecific binding, or signals generated during hybridization or amplification procedures, digital image analysis systems including but not limited to those that detect radioactivity of the probes or chemiluminescence can be employed.

### Screening Assays

The present invention also provides a screen for identifying leads, drugs, therapeutic biologics, and methods for reversing the neoplastic condition of the cells or selectively inhibiting growth or proliferation of the cells described above. In one aspect, the screen identifies lead compounds or biological agents which are useful for the treatment of malignancy, hyperplasia or metaplasia characterized by differential expression of a gene identified in Table 1.

Thus, to practice the method in vitro, suitable cell cultures or tissue cultures are first provided. The cell can be a cultured cell or a genetically modified cell which differentially expresses a gene associated with a neoplastic lung cell e.g., at least one gene identified in Table 1. Alternatively, the cells can be from a tissue biopsy. The cells are cultured under conditions (temperature, growth or culture medium and gas (CO₂)) and for an appropriate amount of time to attain exponential proliferation without density dependent constraints. It also is desirable to maintain an additional separate cell culture; one which does not receive the agent being tested as a control.

As is apparent to one of skill in the art, the method can be modified for high throughput analysis and suitable cells may be cultured in microtiter plates and several agents may be assayed at the same time by noting genotypic changes, phenotypic changes and/or cell death.

When the agent is a composition other than a DNA or RNA nucleic acid molecule, the suitable conditions comprise directly added to the cell culture or added to culture medium for addition. As is apparent to those skilled in the art, an “effective” amount must be added which can be empirically determined.

The screen involves contacting the agent with a test cell characterized by differential expression of a gene of interest and then assaying the cell for the level of said gene expression. In some aspects, it may be necessary to determine the level of gene expression prior to the assay. This provides a base line to compare expression after administration of the agent to the cell culture. In another embodiment, the test cell is a cultured cell from an established cell line that differentially expresses a gene of interest. An agent is a possible therapeutic agent if gene expression is returned (reduced or increased) to a level that is present in a cell in a normal or non-neoplastic state, or the cell selectively dies, or exhibits reduced rate of growth.

In yet another aspect, the test cell or tissue sample is isolated from the subject to be treated and one or more potential agents are screened to determine the optimal therapeutic and/or course of treatment for that individual patient.

For the purposes of this invention, an “agent” is intended to include, but not be limited to a biological or chemical compound such as a simple or complex organic or inorganic molecule, a peptide, a protein or an oligonucleotide. A vast array of compounds can be synthesized, for example oligomers, such as oligopeptides and oligonucleotides, and synthetic organic compounds based on various core structures, and these are also included in the term “agent”. In addition, various natural sources can provide compounds for screening, such as plant or animal extracts, and the like. It should be understood, although not always explicitly stated that the agent is used alone or in combination with another agent, having the same or different biological activity as the agents identified by the inventive screen. The agents and methods also are intended to be combined with other therapies.

As used herein, the term “reversing the neoplastic state of the cell” is intended to include apoptosis, necrosis or any other means of preventing cell division, reduced tumorigenicity, loss of pharmaceutical resistance, maturation, differentiation or reversion of the neoplastic phenotypes as described herein. As noted above, lung cells having differential expression of a gene of interest that results in the neoplastic state are suitably treated by this method. These cells can be identified by any method known in the art that allows for the identification of differential expression of the gene.

When the agent is a nucleic acid, it can be added to the cell cultures by methods known in the art, which includes, but is not limited to calcium phosphate precipitation, microinjection or electroporation. Alternatively or additionally, the nucleic acid can be incorporated into an expression or insertion vector for incorporation into the cells. Vectors that contain both a promoter and a cloning site into which a polynucleotide can be operatively linked are well known in the art and briefly described infra.

Polynucleotides are inserted into vector genomes using methods well known in the art. For example, insert and vector DNA can be contacted, under suitable conditions, with a restriction enzyme to create complementary ends on each molecule that can pair with each other and be joined together with a ligase. Alternatively, synthetic nucleic acid linkers can be ligated to the termini of restricted polynucleotide. These synthetic linkers contain nucleic acid sequences that correspond to a particular restriction site in the vector DNA. Additionally, an oligonucleotide containing a termination codon and an appropriate restriction site can be ligated for insertion into a vector containing, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; versatile multiple cloning sites; and T7 and SP6 RNA promoters for in vitro transcription of sense and antisense RNA. Other means are well known and available in the art.

One can determine if the object of the method, i.e., reversal of the neoplastic state of the cell, has been achieved by a reduction of cell division, differentiation of the cell or assaying for a reduction in gene overexpression. Cellular differentiation can be monitored by histological methods or by monitoring for the presence or loss of certain cell surface markers, which may be associated with an undifferentiated phenotype, e.g., the expression products of at least one gene selected from EGFR-RS, RYK, TNFRSF25, and TRPM7.

Kits containing the agents and instructions necessary to perform the screen and in vitro method as described herein also are claimed.

When the subject is an animal such as a rat or mouse, the method provides a convenient animal model system which can be used prior to clinical testing of the therapeutic agent or alternatively, for lead optimization. In this system, a candidate agent is a potential drug if gene expression is returned to a normal level or if symptoms associated or correlated to the presence of cells containing differential expression of a gene of interest are ameliorated, each as compared to untreated, animal having the pathological cells. It also can be useful to have a separate negative control group of cells or animals which are healthy and not treated, which provides a basis for comparison.

### Therapeutic Methods

Applicants have identified the death domain within the intracellular regions of the gene expression products identified in Table 1 as TNFRSF25, and UNC5H2. Stimulation of receptor proteins containing these motifs results in the induction of apoptosis or cell death. Thus, by contacting the extracellular portion of the receptor with a ligand that binds and activates the receptor, one can initiate apoptotic cell death in lung cancer cells. Such ligands are known in the art and include, but are not limited to polyclonal and monoclonal antibodies as well as small molecules that bind to the extracellular portion of these receptors. Thus, these ligands are useful as therapeutic agents to inhibit growth of cells expressing these receptors.

Applicants have discovered that any one of the gene expression products of the genes identified in Table 1 as EGFR-RS, RYK, TNFRSF25, TRPM7, UNC5H2 and KIAA1883 has sequence similarity to the receptor tyrosine kinase proteins. Receptor tyrosine kinase proteins contain at least seven structural variants. All of the receptor tyrosine kinases are composed of at least three domains: an extracellular glycosylated ligand binding domain, a transmembrane domain and a cytoplasmic catalytic domain that can phosphorylate tyrosine residues. Ligand binding to membrane-bound receptors induces the formation of receptor dimers and allosteric changes that activate the intracellular kinase domains and result in the self-phosphorylation (autophosphorylation and/or transphosphorylation) of the receptor on tyrosine residues. Thus, agents that inhibit receptor activation such as small molecules and antibodies that bind to the receptor's natural ligand are useful to inhibit proliferation of cells expressing these receptors. Polynucleotides expressing these ligands and host cells containing them are also useful as therapeutic agents.

Therapeutic agents also include immune effector cells that specifically recognize and lyse cells expressing a gene identified in Table 1. One can determine if a subject or patient will be beneficially treated by the use of these immune effector cells by screening one or more of the effector cells against tumor cells isolated from the subject or patient using methods known in the art.

In one embodiment, the therapeutic agent is administered in an amount effective to treat lung cancer. In a further preferred embodiment, an agent of the invention is administered in an amount effective to treat non-small cell lung cancer. Therapeutics of the invention can also be used to prevent progression from a pre-neoplastic or non-malignant state into a neoplastic or a malignant state.

Various delivery systems are known and can be used to administer a therapeutic agent of the invention, e.g., encapsulation in liposomes, microparticles, microcapsules, expression by recombinant cells, receptor-mediated endocytosis (See, e.g., Wu and Wu, (1987), J. Biol. Chem. 262:4429-4432), construction of a therapeutic nucleic acid as part of a retroviral or other vector, etc. Methods of delivery include but are not limited to intra-arterial, intra-muscular, intravenous, intranasal, and oral routes. In a specific embodiment, it may be desirable to administer the pharmaceutical compositions of the invention locally to the area in need of treatment; this may be achieved by, for example, and not by way of limitation, local infusion during surgery, by injection, or by means of a catheter.

The agents identified herein as effective for their intended purpose can be administered to subjects or individuals susceptible to or at risk of developing a disease correlated to the differential expression of a gene of Table 1. When the agent is administered to a subject such as a mouse, a rat or a human patient, the agent can be added to a pharmaceutically acceptable carrier and systemically or topically administered to the subject. To determine patients that can be beneficially treated, a tumor sample is removed from the patient and the cells are assayed for the differential expression of the gene. Therapeutic amounts can be empirically determined and will vary with the pathology being treated, the subject being treated and the efficacy and toxicity of the agent. When delivered to an animal, the method is useful to further confirm efficacy of the agent. As an example of an animal model, groups of nude mice (Balb/c NCR nu/nu female, Simonsen, Gilroy, Calif.) are each subcutaneously inoculated with about 10⁵ to about 10⁹ hyperproliferative, cancer or target cells as defined herein. When the tumor is established, the agent is administered, for example, by subcutaneous injection around the tumor. Tumor measurements to determine reduction of tumor size are made in two dimensions using venier calipers twice a week. Other animal models may also be employed as appropriate.

Administration in vivo can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents can be found below.

The agents and compositions of the present invention can be used in the manufacture of medicaments and for the treatment of humans and other animals by administration in accordance with conventional procedures, such as an active ingredient in pharmaceutical compositions.

The pharmaceutical compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to an agent of the present invention, the pharmaceutical compositions can also contain other pharmaceutically active compounds or a plurality of compounds of the invention.

More particularly, an agent of the present invention also referred to herein as the active ingredient, may be administered for therapy by any suitable route including oral, rectal, nasal, topical (including transdermal, aerosol, buccal and sublingual), vaginal, parental (including subcutaneous, intramuscular, intravenous and intradermal) and pulmonary. It will also be appreciated that the preferred route will vary with the condition and age of the recipient, and the disease being treated.

Ideally, the agent should be administered to achieve peak concentrations of the active compound at sites of disease. This may be achieved, for example, by the intravenous injection of the agent, optionally in saline, or orally administered, for example, as a tablet, capsule or syrup containing the active ingredient. Desirable blood levels of the agent may be maintained by a continuous infusion to provide a therapeutic amount of the active ingredient within disease tissue. The use of operative combinations is contemplated to provide therapeutic combinations requiring a lower total dosage of each component antiviral agent than may be required when each individual therapeutic compound or drug is used alone, thereby reducing adverse effects.

While it is possible for the agent to be administered alone, it is preferable to present it as a pharmaceutical formulation comprising at least one active ingredient, as defined above, together with one or more pharmaceutically acceptable carriers therefor and optionally other therapeutic agents. Each carrier must be “acceptable” in the sense of being compatible with the other ingredients of the formulation and not injurious to the patient.

Formulations include those suitable for oral, rectal, nasal, topical (including transdermal, buccal and sublingual), vaginal, parenteral (including subcutaneous, intramuscular, intravenous and intradermal) and pulmonary administration. The formulations may conveniently be presented in unit dosage form and may be prepared by any methods well known in the art of pharmacy. Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then if necessary shaping the product.

Formulations of the present invention suitable for oral administration may be presented as discrete units such as capsules, cachets or tablets, each containing a predetermined amount of the active ingredient; as a powder or granules; as a solution or suspension in an aqueous or non-aqueous liquid; or as an oil-in-water liquid emulsion or a water-in-oil liquid emulsion. The active ingredient may also be presented a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions for topical administration according to the present invention may be formulated as an ointment, cream, suspension, lotion, powder, solution, past, gel, spray, aerosol or oil. Alternatively, a formulation may comprise a patch or a dressing such as a bandage or adhesive plaster impregnated with active ingredients and optionally one or more excipients or diluents.

If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the agent through the skin or other affected areas. Examples of such dermal penetration enhancers include dimethylsulfoxide and related analogues.

The oily phase of the emulsions of this invention may be constituted from known ingredients in a known manner. While this phase may comprise merely an emulsifier (otherwise known as an emulgent), it desirably comprises a mixture of at lease one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulphate.

The choice of suitable oils or fats for the formulation is based on achieving the desired cosmetic properties, since the solubility of the active compound in most oils likely to be used in pharmaceutical emulsion formulations is very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the agent.

Formulations for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the agent, such carriers as are known in the art to be appropriate.

Formulations suitable for nasal administration, wherein the carrier is a solid, include a coarse powder having a particle size, for example, in the range of about 20 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the agent.

Formulations suitable for parenteral administration include aqueous and non-aqueous isotonic sterile injection solutions which may contain anti-oxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily subdose, as herein above-recited, or an appropriate fraction thereof, of an agent.

It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies.

### Transgenic Animals

In another aspect, the genes of Table 1 can be used to generate transgenic animal models. In recent years, geneticists have succeeded in creating transgenic animals, for example mice, by manipulating the genes of developing embryos and introducing foreign genes into these embryos. Once these genes have integrated into the genome of the recipient embryo, the resulting embryos or adult animals can be analyzed to determine the function of the gene. The mutant animals are produced to understand the function of known genes in vivo and to create animal models of human diseases. (See, e.g., Chisaka et al. (1992) 355:516-520; Joyner et al. (1992) in POSTIMPLANTATION DEVELOPMENT IN THE MOUSE (Chadwick and Marsh, eds., John Wiley & Sons, United Kingdom) pp:277-297; Dorin et al. (1992) Nature 359:211-215).

U.S. Pat. Nos. 5,464,764 and 5,487,992 describe one type of transgenic animal in which the gene of interest is deleted or mutated sufficiently to disrupt its function. (See, also U.S. Pat. Nos. 5,631,153 and 5,627,059). These “knock-out” animals, made by taking advantage of the phenomena of homologous recombination, can be used to study the function of a particular gene sequence in vivo. The polynucleotide sequences described herein are useful in preparing animal models of lung cancer.

### Antibodies

Also provided by this invention is an antibody capable of specifically forming a complex with the expression product of a gene of interest. The term “antibody” includes polyclonal antibodies and monoclonal antibodies. The antibodies include, but are not limited to mouse, rat, and rabbit or human antibodies. The antibodies are useful to identify and purify gene expression products as well as APCs expressing the polypeptides.

Laboratory methods for producing polyclonal antibodies and monoclonal antibodies, as well as deducing their corresponding nucleic acid sequences, are known in the art, see Harlow and Lane (1988) supra and Sambrook et al. (1989) supra. The monoclonal antibodies of this invention can be biologically produced by introducing protein or a fragment thereof into an animal, e.g., a mouse or a rabbit. The antibody producing cells in the animal are isolated and fused with myeloma cells or hetero-myeloma cells to produce hybrid cells or hybridomas. Accordingly, the hybridoma cells producing the monoclonal antibodies of this invention also are provided.

Thus, using the protein or fragment thereof, and well known methods, one of skill in the art can produce and screen the hybridoma cells and antibodies of this invention for antibodies having the ability to bind the proteins or polypeptides.

If a monoclonal antibody being tested binds with the protein or polypeptide, then the antibody being tested and the antibodies provided by the hybridomas of this invention are equivalent. It also is possible to determine without undue experimentation, whether an antibody has the same specificity as the monoclonal antibody of this invention by determining whether the antibody being tested prevents a monoclonal antibody of this invention from binding the protein or polypeptide with which the monoclonal antibody is normally reactive. If the antibody being tested competes with the monoclonal antibody of the invention as shown by a decrease in binding by the monoclonal antibody of this invention, then it is likely that the two antibodies bind to the same or a closely related epitope. Alternatively, one can pre-incubate the monoclonal antibody of this invention with a protein with which it is normally reactive, and determine if the monoclonal antibody being tested is inhibited in its ability to bind the antigen. If the monoclonal antibody being tested is inhibited then, in all likelihood, it has the same, or a closely related, epitopic specificity as the monoclonal antibody of this invention.

The term “antibody” also is intended to include antibodies of all isotypes. Particular isotypes of a monoclonal antibody can be prepared either directly by selecting from the initial fusion, or prepared secondarily, from a parental hybridoma secreting a monoclonal antibody of different isotype by using the sib selection technique to isolate class switch variants using the procedure described in Steplewski et al. (1985) Proc. Natl. Acad. Sci. USA 82:8653 or Spira et al. (1984) J. Immunol. Meth. 74:307.

This invention also provides biological active fragments of the polyclonal and monoclonal antibodies described above. These “antibody fragments” retain some ability to selectively bind with its antigen or immunogen. Such antibody fragments can include, but are not limited to:
(1) Fab,
(2) Fab′,
(3) F(ab′)₂,
(4) Fv, and
(5) SCA

A specific example of “a biologically active antibody fragment” is a CDR region of the antibody. Methods of making these fragments are known in the art, see for example, Harlow and Lane (1988) supra.

The antibodies of this invention also can be modified to create chimeric antibodies and humanized antibodies (Oi et al. (1986) BioTechniques 4(3):214). Chimeric antibodies are those in which the various domains of the antibodies' heavy and light chains are coded for by DNA from more than one species.

The isolation of other hybridomas secreting monoclonal antibodies with the specificity of the monoclonal antibodies of the invention can also be accomplished by one of ordinary skill in the art by producing anti-idiotypic antibodies (Herlyn et al. (1986) Science 232:100). An anti-idiotypic antibody is an antibody which recognizes unique determinants present on the monoclonal antibody produced by the hybridoma of interest.

Idiotypic identity between monoclonal antibodies of two hybridomas demonstrates that the two monoclonal antibodies are the same with respect to their recognition of the same epitopic determinant. Thus, by using antibodies to the epitopic determinants on a monoclonal antibody it is possible to identify other hybridomas expressing monoclonal antibodies of the same epitopic specificity.

It is also possible to use the anti-idiotype technology to produce monoclonal antibodies which mimic an epitope. For example, an anti-idiotypic monoclonal antibody made to a first monoclonal antibody will have a binding domain in the hypervariable region which is the mirror image of the epitope bound by the first monoclonal antibody. Thus, in this instance, the anti-idiotypic monoclonal antibody could be used for immunization for production of these antibodies.

As used in this invention, the term “epitope” is meant to include any determinant having specific affinity for the monoclonal antibodies of the invention. Epitopic determinants usually consist of chemically active surface groupings of molecules such as amino acids or sugar side chains and usually have specific three dimensional structural characteristics, as well as specific charge characteristics.

The antibodies of this invention can be linked to a detectable agent or label. There are many different labels and methods of labeling known to those of ordinary skill in the art.

The coupling of antibodies to low molecular weight haptens can increase the sensitivity of the assay. The haptens can then be specifically detected by means of a second reaction. For example, it is common to use haptens such as biotin, which reacts avidin, or dinitrophenol, pyridoxal, and fluorescein, which can react with specific anti-hapten antibodies. See, Harlow and Lane (1988) supra.

The monoclonal antibodies of the invention also can be bound to many different carriers. Thus, this invention also provides compositions containing the antibodies and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such, using routine experimentation.

Compositions containing the antibodies, fragments thereof or cell lines which produce the antibodies, are encompassed by this invention. When these compositions are to be used pharmaceutically, they are combined with a pharmaceutically acceptable carrier.

### Antigen-presenting Cells

In another embodiment the present invention provides a method of inducing an immune response comprising delivering the compounds and compositions of the invention in the context of an MHC molecule. Thus, the polypeptides of this invention can be pulsed into antigen presenting cells using the methods described herein. Antigen-presenting cells, include, but are not limited to dendritic cells (DCs), monocytes/macrophages, B lymphocytes or other cell type(s) expressing the necessary MHC/co-stimulatory molecules. The methods described below focus primarily on DCs which are the most potent, preferred APCs. These host cells containing the polypeptides or proteins are further provided.

Isolated host cells which present the polypeptides of this invention in the context of MHC molecules are further useful to expand and isolate a population of educated, antigen-specific immune effector cells. The immune effector cells, e.g., cytotoxic T lymphocytes, are produced by culturing naïve immune effector cells with antigen-presenting cells which present the polypeptides in the context of MHC molecules on the surface of the APCs. The population can be purified using methods known in the art, e.g., FACS analysis or ficoll gradient. The methods to generate and culture the immune effector cells as well as the populations produced thereby also are the inventor's contribution and invention. Pharmaceutical compositions comprising the cells and pharmaceutically acceptable carriers are useful in adoptive immunotherapy. Prior to administration in vivo, the immune effector cells are screened in vitro for their ability to lyse tumor cells

In one embodiment, the immune effector cells and/or the APCs are genetically modified. Using standard gene transfer, genes coding for co-stimulatory molecules and/or stimulatory cytokines can be inserted prior to, concurrent to or subsequent to expansion of the immune effector cells.

This invention also provides methods of inducing an immune response in a subject, comprising administering to the subject an effective amount of a polypeptide described above under the conditions that induce an immune response to the polypeptide. The polypeptide can be administered in a formulation or as a polynucleotide encoding the polypeptide. The polynucleotide can be administered in a gene delivery vehicle or by inserting into a host cell which in turn recombinantly transcribes, translates and processed the encoded polypeptide. Isolated host cells containing the polynucleotides of this invention in a pharmaceutically acceptable carrier can therefore be combined with appropriate and effective amount of an adjuvant, cytokine or co-stimulatory molecule for an effective vaccine regimen. In one embodiment, the host cell is an APC such as a dendritic cell. The host cell can be further modified by inserting of a polynucleotide coding for an effective amount of either or both a cytokine and/or a co-stimulatory molecule.

The methods of this invention can be further modified by co-administering an effective amount of a cytokine or co-stimulatory molecule to the subject.

This invention also provides compositions containing any of the above-mentioned proteins, polypeptides, polynucleotides, vectors, cells, antibodies and fragments thereof, and an acceptable solid or liquid carrier. When the compositions are used pharmaceutically, they are combined with a “pharmaceutically acceptable carrier” for diagnostic and therapeutic use.

### Isolation, Culturing and Expansion of APCs, Including Dendritic Cells

The following is a brief description of two fundamental approaches for the isolation of APC. These approaches involve (1) isolating bone marrow precursor cells (CD34⁺) from blood and stimulating them to differentiate into APC; or (2) collecting the precommitted APCs from peripheral blood. In the first approach, the patient must be treated with cytokines such as GM-CSF to boost the number of circulating CD34⁺ stem cells in the peripheral blood.

The second approach for isolating APCs is to collect the relatively large numbers of precommitted APCs already circulating in the blood. Previous techniques for isolating committed APCs from human peripheral blood have involved combinations of physical procedures such as metrizamide gradients and adherence/non-adherence steps (Freudenthal P. S. et al. (1990) Proc. Natl. Acad. Sci. USA 87:7698-7702); Percoll gradient separations (Mehta-Damani et al. (1994) J. Immunol. 153:996-1003); and fluorescence activated cell sorting techniques (Thomas R. et al. (1993) J. Immunol. 151:6840-6852).

One technique for separating large numbers of cells from one another is known as countercurrent centrifugal elutriation (CCE). In this technique, cells are subject to simultaneous centrifugation and a washout stream of buffer that is constantly increasing in flow rate. The constantly increasing countercurrent flow of buffer leads to fractional cell separations that are largely based on cell size.

In one aspect of the invention, the APC are precommitted or mature dendritic cells which can be isolated from the white blood cell fraction of a mammal, such as a murine, simian or a human (See, e.g., WO 96/23060). The white blood cell fraction can be from the peripheral blood of the mammal. This method includes the following steps: (a) providing a white blood cell fraction obtained from a mammalian source by methods known in the art such as leukapheresis; (b) separating the white blood cell fraction of step (a) into four or more subfractions by countercurrent centrifugal elutriation; (c) stimulating conversion of monocytes in one or more fractions from step (b) to dendritic cells by contacting the cells with calcium ionophore, GM-CSF and IL-13 or GM-CSF and IL-4, (d) identifying the dendritic cell-enriched fraction from step (c); and (e) collecting the enriched fraction of step (d), is performed at about 4° C. One way to identify the dendritic cell-enriched fraction is by fluorescence-activated cell sorting. The white blood cell fraction can be treated with calcium ionophore in the presence of other cytokines, such as recombinant (rh) rhIL-12, rhGM-CSF, or rhIL-4. The cells of the white blood cell fraction can be washed in buffer and suspended in Ca⁺⁺/Mg⁺⁺ free media prior to the separating step. The white blood cell fraction can be obtained by leukapheresis. The dendritic cells can be identified by the presence of at least one of the following markers: HLA-DR, HLA-DQ, or B7.2, and the simultaneous absence of the following markers: CD3, CD14, CD16, 56, 57, and CD 19, 20. Monoclonal antibodies specific to these cell surface markers are commercially available.

More specifically, the method requires collecting an enriched collection of white cells and platelets from leukapheresis that is then further fractionated by countercurrent centrifugal elutriation (CCE) (Abrahamsen T. G. et al. (1991) J. Clin. Apheresis. 6:48-53). Cell samples are placed in a special elutriation rotor. The rotor is then spun at a constant speed of, for example, 3000 rpm. Once the rotor has reached the desired speed, pressurized air is used to control the flow rate of cells. Cells in the elutriator are subjected to simultaneous centrifugation and a washout stream of buffer that is constantly increasing in flow rate. This results in fractional cell separations based largely but not exclusively on differences in cell size.

Quality control of APC and more specifically DC collection and confirmation of their successful activation in culture is dependent upon a simultaneous multi-color FACS analysis technique which monitors both monocytes and the dendritic cell subpopulation as well as possible contaminant T lymphocytes. It is based upon the fact that DCs do not express the following markers: CD3 (T cell); CD14 (monocyte); CD16, 56, 57 (NK/LAK cells); CD19, 20 (B cells). At the same time, DCs do express large quantities of HLA-DR, significant HLA-DQ and B7.2 (but little or no B7.1) at the time they are circulating in the blood (in addition they express Leu M7 and M9, myeloid markers which are also expressed by monocytes and neutrophils).

When combined with a third color reagent for analysis of dead cells, propidium iodide (PI), it is possible to make positive identification of all cell subpopulations (see Table 2):

TABLE 2
FACS analysis of fresh peripheral cell subpopulations

| | Color #1 | | |
|---|---|---|---|
| | Cocktail | Color #2 | Color #3 |
| | 3/14/16/19/20/56/57 | HLA-DR | PI |
| LiveDendritic cells | Negative | Positive | Negative |
| Live Monocytes | Positive | Positive | Negative |
| Live Neutrophils | Negative | Negative | Negative |
| Dead Cells | Variable | Variable | Positive |

Additional markers can be substituted for additional analysis:

Color #1: CD3 alone, CD14 alone, etc.; Leu M7 or Leu M9; anti-Class 1, etc.

Color #2: HLA-DQ, B7.1, B7.2, CD25 (IL2r), ICAM, LFA-3, etc.

The goal of FACS analysis at the time of collection is to confirm that the DCs are enriched in the expected fractions, to monitor neutrophil contamination, and to make sure that appropriate markers are expressed. This rapid bulk collection of enriched DCs from human peripheral blood, suitable for clinical applications, is absolutely dependent on the analytic FACS technique described above for quality control. If need be, mature DCs can be immediately separated from monocytes at this point by fluorescent sorting for “cocktail negative” cells. It may not be necessary to routinely separate DCs from monocytes because, as will be detailed below, the monocytes themselves are still capable of differentiating into DCs or functional DC-like cells in culture.

Once collected, the DC rich/monocyte APC fractions (usually 150 through 190) can be pooled and cryopreserved for future use, or immediately placed in short term culture.

Alternatively, others have reported a method for upregulating (activating) dendritic cells and converting monocytes to an activated dendritic cell phenotype. This method involves the addition of calcium ionophore to the culture media to convert monocytes into activated dendritic cells. Adding the calcium ionophore A23187, for example, at the beginning of a 24 to 48 hour culture period resulted in uniform activation and dendritic cell phenotypic conversion of the pooled “monocyte plus DC” fractions: characteristically, the activated population becomes uniformly CD14 (Leu M3) negative, and upregulates HLA-DR, HLA-DQ, ICAM-1, B7.1, and B7.2. Furthermore, this activated bulk population functions as well on a small numbers basis and is easily purified.

Specific combination(s) of cytokines have been used successfully to amplify (or partially substitute) for the activation/conversion achieved with calcium ionophore: these cytokines include but are not limited to purified or recombinant (“rh”) rhGM-CSF, rhIL-2, and rhIL-4. Each cytokine when given alone is inadequate for optimal upregulation.

### Presentation of Antigen to the APC

Polypeptides expressed from the genes of Table 1, can be delivered to antigen-presenting cells as protein/peptide or in the form of cDNA encoding the protein/peptide. Antigen-presenting cells (APCs) can consist of dendritic cells (DCs), monocytes/macrophages, B lymphocytes or other cell type(s) expressing the necessary MHC/co-stimulatory molecules. The methods described below focus primarily on DCs which are the most potent, preferred APCs.

Pulsing is accomplished in vitro/ex vivo by exposing APCs to the antigenic protein or peptide(s) of this invention. The protein or peptide(s) is added to APCs at a concentration of 1-10 μm for approximately 3 hours. Pulsed APCs can subsequently be administered to the host via an intravenous, subcutaneous, intranasal, intramuscular or intraperitoneal route of delivery.

Protein/peptide antigen can also be delivered in vivo with adjuvant via the intravenous, subcutaneous, intranasal, intramuscular or intraperitoneal route of delivery.

Paglia et al. (1996) J. Exp. Med. 183:317-322, has shown that APC incubated with whole protein in vitro were recognized by MHC class I-restricted CTLs, and that immunization of animals with these APCs led to the development of antigen-specific CTLs in vivo. In addition, several different techniques have been described which lead to the expression of antigen in the cytosol of APCs, such as DCs. These include (1) the introduction into the APCs of RNA isolated from tumor cells, (2) infection of APCs with recombinant vectors to induce endogenous expression of antigen, and (3) introduction of tumor antigen into the DC cytosol using liposomes. (See Boczkowski D. et al. (1996) J. Exp. Med. 184:465-472; Rouse et al. (1994) J. Virol. 68:5685-5689; and Nair et al. (1992) J. Exp. Med. 175:609-612).

### Foster Antigen Presenting Cells

Foster APCs are derived from the human cell line 174xCEM.T2, referred to as T2, which contains a mutation in its antigen processing pathway that restricts the association of endogenous peptides with cell surface MHC class I molecules (Zweerink et al. (1993) J. Immunol. 150:1763-1771). This is due to a large homozygous deletion in the MHC class II region encompassing the genes TAP1, TAP2, LMP1, and LMP2, which are required for antigen presentation to MHC class 1-restricted CD8⁺ CTLs. In effect, only “empty” MHC class I molecules are presented on the surface of these cells. Exogenous peptide added to the culture medium binds to these MHC molecules provided that the peptide contains the allele-specific binding motif. These T2 cells are referred to herein as “foster” APCs. They can be used in conjunction with this invention to present antigen(s).

Transduction of T2 cells with specific recombinant MHC alleles allows for redirection of the MHC restriction profile. Libraries tailored to the recombinant allele will be preferentially presented by them because the anchor residues will prevent efficient binding to the endogenous allele.

High level expression of MHC molecules makes the APC more visible to the CTLs. Expressing the MHC allele of interest in T2 cells using a powerful transcriptional promoter (e.g., the CMV promoter) results in a more reactive APC (most likely due to a higher concentration of reactive MHC-peptide complexes on the cell surface).

### Immunogenicity Assays

The immunogenicity of therapeutic agents of this invention can be determined by known methodologies including, but not limited to those exemplified below. In one embodiment, such methodology may be employed to compare an equivalent polypeptide ligand of the invention with the corresponding native ligand. For example, an altered ligand may be considered “more active” if it compares favorably with the activity of the native ligand in any one of the following assays. For some purposes, one skilled in the art will select an immunogenic ligand which displays more activity than another immunogenic ligand, i.e., for treatment and/or diagnostic purposes. However, for some applications, the use of an immunogenic ligand which is comparable with the native ligand will be suitable. In other situations, it may be desirable to utilize an immunogenic ligand which is less active. It has been suggested that such levels of activity positively correlate with the level of immunogenicity.

⁵¹Cr-release lysis assay. Lysis of peptide-pulsed ⁵¹Cr-labeled targets by antigen-specific T cells can be compared for target cells pulsed with either the native or altered ligands. Functionally enhanced ligands will show greater lysis of targets as a function of time. The kinetics of lysis as well as overall target lysis at a fixed timepoint (e.g., 4 hours) may be used to evaluate ligand performance. (Ware C. F. et al. (1983) J. Immunol. 131:1312).

Cytokine-release assay. Analysis of the types and quantities of cytokines secreted by T cells upon contacting ligand-pulsed targets can be a measure of functional activity. Cytokines can be measured by ELISA or ELISPOT assays to determine the rate and total amount of cytokine production. (Fujihashi K. et al. (1993) J. Immunol. Meth. 160:181; Tanguay S. and Killion J. J. (1994) Lymphokine Cytokine Res. 13:259).

In vitro T cell education. The ligands of the invention can be compared to the corresponding native ligand for the ability to elicit ligand-reactive T cell populations from normal donor or patient-derived PBMC. In this system, elicited T cells can be tested for lytic activity, cytokine-release, polyclonality, and cross-reactivity to the native ligand. (Parkhurst M. R. et al. (1996) J. Immunol. 157:2539).

Transgenic animal models. Immunogenicity can be assessed in vivo by vaccinating HLA transgenic mice with either the ligands of the invention or the native ligand and determining the nature and magnitude of the induced immune response. Alternatively, the hu-PBL-SCID mouse model allows reconstitution of a human immune system in a mouse by adoptive transfer of human PBL. These animals may be vaccinated with the ligands and analyzed for immune response as previously mentioned. (Shirai M. et al. (1995) J. Immunol. 154:2733; Mosier D. E. et al. (1993) Proc. Natl. Acad. Sci. USA 90:2443).

Proliferation. T cells will proliferate in response to reactive ligands. Proliferation can be monitored quantitatively by measuring, for example, ³H-thymidine uptake. (Caruso A. et al. (1997) Cytometry 27:71).

Tetramer staining. MHC tetramers can be loaded with individual ligands and tested for their relative abilities to bind to appropriate effector T cell populations. (Altman J. D. et al. (1996) Science 274(5284):94-96).

MHC Stabilization. Exposure of certain cell lines such as T2 cells to HLA-binding ligands results in the stabilization of MHC complexes on the cell surface. Quantitation of MHC complexes on the cell surface has been correlated with the affinity of the ligand for the HLA allele that is stabilized. Thus, this technique can determine the relative HLA affinity of ligand epitopes. (Stuber G. et al. (1995) Int. Immunol. 7:653).

MHC competition. The ability of a ligand to interfere with the functional activity of a reference ligand and its cognate T cell effectors is a measure of how well a ligand can compete for MHC binding. Measuring the relative levels of inhibition is an indicator of MHC affinity. (Feltkamp M. C. et al. (1995) Immunol. Lett. 47:1).

Primate models. A recently described non-human primate (chimpanzee) model system can be utilized to monitor in vivo immunogenicities of HLA-restricted ligands. It has been demonstrated that chimpanzees share overlapping MHC-ligand specificities with human MHC molecules thus allowing one to test HLA-restricted ligands for relative in vivo immunogenicity. (Bertoni R. et al. (1998) J. Immunol. 161:4447).

Monitoring TCR Signal Transduction Events. Several intracellular signal transduction events (e.g., phosphorylation) are associated with successful TCR engagement by MHC-ligand complexes. The qualitative and quantitative analysis of these events have been correlated with the relative abilities of ligands to activate effector cells through TCR engagement. (Salazar E. et al. (2000) Int. J. Cancer 85:829; Isakov N. et al. (1995) J. Exp. Med. 181:375).

### Expansion of Immune Effector Cells

The present invention makes use of these APCs to stimulate production of an enriched population of antigen-specific immune effector cells. The antigen-specific immune effector cells are expanded at the expense of the APCs, which die in the culture. The process by which naïve immune effector cells become educated by other cells is described essentially in Coulie (1997) Molec. Med. Today 3:261-268.

The APCs prepared as described above are mixed with naïve immune effector cells. The cells may be cultured in the presence of a cytokine, for example IL-2. Because dendritic cells secrete potent immunostimulatory cytokines, such as IL-12, it may not be necessary to add supplemental cytokines during the first and successive rounds of expansion. In any event, the culture conditions are such that the antigen-specific immune effector cells expand (i.e., proliferate) at a much higher rate than the APCs. Multiple infusions of APCs and optional cytokines can be performed to further expand the population of antigen-specific cells.

In one embodiment, the immune effector cells are T cells. In a separate embodiment, the immune effector cells can be genetically modified by transduction with a transgene coding for example, IL-2, IL-11 or IL-13. Methods for introducing transgenes in vitro, ex vivo and in vivo are known in the art. See Sambrook et al. (1989) supra.

APCs can be transduced with viral vectors encoding a relevant polypeptides. The most common viral vectors include recombinant poxviruses such as vaccinia and fowlpox virus (Bronte et al. (1997) Proc. Natl. Acad. Sci. USA 94:3183-3188; Kim et al. (1997) J. Immunother. 20:276-286) and as an example adenovirus (Arthur et al. (1997) J. Immunol. 159:1393-1403; Wan et al. (1997) Human Gene Therapy 8:1355-1363; Huang et al. (1995) J. Virol. 69:2257-2263). Retrovirus also may be used for transduction of human APCs (Marin et al. (1996) J. Virol. 70:2957-2962).

In vitro/ex vivo, exposure of human DCs to adenovirus (Ad) vector at a multiplicity of infection (MOI) of 500 for 16-24 h in a minimal volume of serum-free medium reliably gives rise to transgene expression in 90-100% of DCs. The efficiency of transduction of DCs or other APCs can be assessed by immunofluorescence using fluorescent antibodies specific for the tumor antigen being expressed (Kim et al. (1997) J. Immunother. 20:276-286). Alternatively, the antibodies can be conjugated to an enzyme (e.g., HRP) giving rise to a colored product upon reaction with the substrate. The actual amount of antigenic polypeptides being expressed by the APCs can be evaluated by ELISA.

Transduced APCs can subsequently be administered to the host via an intravenous, subcutaneous, intranasal, intramuscular or intraperitoneal route of delivery.

In vivo transduction of DCs, or other APCs, can be accomplished by administration of Ad (or other viral vectors) via different routes including intravenous, intramuscular, intranasal, intraperitoneal or cutaneous delivery. In one embodiment, the method is cutaneous delivery of Ad vector at multiple sites using a total dose of approximately 1×10¹⁰−1×10¹² i.u. Levels of in vivo transduction can be roughly assessed by co-staining with antibodies directed against APC marker(s) and the TAA being expressed. The staining procedure can be carried out on biopsy samples from the site of administration or on cells from draining lymph nodes or other organs where APCs (in particular DCs) may have migrated (Condon et al. (1996) Nature Med. 2:1122-1128 and Wan et al. (1997) Hum. Gene Ther. 8:1355-1363). The amount of antigen being expressed at the site of injection or in other organs where transduced APCs may have migrated can be evaluated by ELISA on tissue homogenates.

Although viral gene delivery is more efficient, DCs can also be transduced in vitro/ex vivo by non-viral gene delivery methods such as electroporation, calcium phosphate precipitation or cationic lipid/plasmid DNA complexes (Arthur et al. (1997) Cancer Gene Ther. 4:17-25). Transduced APCs can subsequently be administered to the host via an intravenous, subcutaneous, intranasal, intramuscular or intraperitoneal route of delivery.

In vivo transduction of DCs, or other APCs, can potentially be accomplished by administration of cationic lipid/plasmid DNA complexes delivered via the intravenous, intramuscular, intranasal, intraperitoneal or cutaneous route of administration. Gene gun delivery or injection of naked plasmid DNA into the skin also leads to transduction of DCs (Condon et al. (1996) Nature Med. 2:1122-1128; Raz et al. (1994) Proc. Natl. Acad. Sci. USA 91:9519-9523). Intramuscular delivery of plasmid DNA may also be used for immunization (Rosato et al. (1997) Hum. Gene Ther. 8:1451-1458.)

The transduction efficiency and levels of transgene expression can be assessed as described above for viral vectors.

### Adoptive Immunotherapy and Vaccines

The expanded populations of antigen-specific immune effector cells of the present invention also find use in adoptive immunotherapy regimes and as vaccines.

Adoptive immunotherapy methods involve, in one aspect, administering to a subject a substantially pure population of educated, antigen-specific immune effector cells made by culturing naïve immune effector cells with APCs as described above. Preferably, the APCs are dendritic cells.

In one embodiment, the adoptive immunotherapy methods described herein are autologous. In this case, the APCs are made using parental cells isolated from a single subject. The expanded population also employs T cells isolated from that subject. Finally, the expanded population of antigen-specific cells is administered to the same patient.

In a further embodiment an effective amount, APCs or immune effector cells are administered with an effective amount of a stimulatory cytokine, such as IL-2 or a co-stimulatory molecule.

The agents identified herein as effective for their intended purpose can be administered to subjects in need of such therapy. Method for administration of therapeutic agents are known in the art and described briefly, supra.

It is to be understood that while the invention has been described in conjunction with the above embodiments, that the foregoing description and the following examples are intended to illustrate and not limit the scope of the invention. Other aspects, advantages and modifications within the scope of the invention will be apparent to those skilled in the art to which the invention pertains.

1. A method of aiding in the diagnosis of the neoplastic condition of a lung cell, comprising detecting at least one gene selected from the group comprising EGFR-RS, RYK, TNFRSF25, TRPM7, UNC5H2, KCP3 and KIAA 1883, that is expressed in a sample, wherein the amount expressed is indicative of the neoplastic condition of the lung cell.

2. The method of claim 1, wherein the amount expressed is higher in the neoplastic condition as compared to a normal cell.

3. The method of claim 1, wherein the amount expressed is less in a neoplastic cell as compared to a normal cell.

4. The method of claim 1, wherein the amount of gene expressed is determined by detecting the quantity of mRNA transcribed from the gene.

5. The method of claim 1, wherein the amount expressed is determined by detecting the quantity of cDNA produced from the reverse transcription of the mRNA transcribed from the gene.

6. The method of claim 1, wherein the amount expressed is determined by detecting the quantity of the polypeptide or protein encoded by the gene.

7. The method of claim 1, wherein the lung cancer is non-small cell lung cancer.

8. A ligand that specifically recognizes and bind a gene expression product or fragment thereof, wherein said gene expression product is at least one of the group EGFR-RS; RYK; TNFRS25; TRPM7; UNC5H2, KCP3 AND KIAA 1883.

9. The ligand of claim 8, wherein said ligand is an antibody or fragment thereof.

10. The ligand of claim 9, wherein said antibody is a monoclonal antibody or a polyclonal antibody.

11. The ligand of claim 10, further comprising a agent selected from the group consisting of a toxin, a detectable label, an adjuvant, a delivery vector and a radioisotopic label.

12. The ligand of claim 10, further comprising a toxin.

13. A screen for a potential therapeutic agent for the reversal of the neoplastic condition of a lung cell wherein the cell is characterized by differential expression of at least one gene selected from the group identified in Table 1, comprising contacting a sample with an effective amount of a potential agent and assaying for reversal of the neoplastic condition.

14. A method for reversing the neoplastic condition of a lung cell, wherein the cell is characterized by differential expression of at least one gene identified in Table 1, comprising contacting the cell with an agent identified by the method of claim 13.

15. A method for inhibiting the growth of a neoplastic lung cell, comprising contacting the cell with a labeled antibody that specifically recognizes and binds a protein expressed on the surface of a cell, wherein the protein is the expression product of a gene selected from the group consisting of is at least one of EGFR-RS, RYK, TNFRSF25, TRPM7, KCP3 and KIAA 1883, thereby inhibiting the growth of the neoplastic lung cell.

16. The method of claim 1, wherein the lung cell is a NSCLC cell.

17. The method of claim 15, wherein the label is a cytotoxic agent.

18. A method for inhibiting the growth of a neoplastic lung cell, comprising contacting the cell with an effective amount of an immune effector cell that specifically recognized and lyses a cell expressing at least one gene selected from the group comprising EGFR-RS, RYK, TNFRSF25, TRPM7, UNC5H2, KCP3 and KIAA 1883, thereby inhibiting the growth of the neoplastic lung cell.

19. A method of inhibiting the growth of a suitable neoplastic lung cell, comprising contacting the cell with an effective amount of an agent that induces apoptosis in the cell.


SEQUENCE LISTING (14 sequences)

SEQ ID NO: 1 - DNA, 3053 bp, Homo sapiens
ctcggcgcgggcgccctcccggccagcggcggcagcccctcctccccggcgccctcaggaccccccagagacccccggcggcggcagcctgccttgctctgccaggaaccatgagtgaggcccgcagggacagcacgagcagcctgcagcgcaagaagccaccctggctaaagctggacattccctctgcggtgcccctgacggcagaagagcccagcttcctgcagcccctgaggcgacaggctttcctgaggagtgtgagtatgccagccgagacagcccacatctcttcaccccaccatgagctccggcggccggtgctgcaacgccagacgtccatcacacagaccatccgcagggggaccgccgactggtttggagtgagcaaggacagtgacagcacccagaaatggcagcgcaagagcatccgtcactgcagccagcgctacgggaagctgaagccccaggtcctccgggagctggacctgcccagccaggacaacgtgtcgctgaccagcaccgagacgccacccccactctacgtggggccatgccagctgggcatgcagaagatcatagaccccctggcccgtggccgtgccttccgtgtggcagatgacactgcggaaggcctgagtgccccacacactcccgtcacgccgggtgctgcctccctctgctccttctccagctcccgctcaggtttccaccggctcccgcggcggcgcaagcgagagtcggtggccaagatgagcttccgggcggccgcagcgctgatgaaaggccgctccgttagggatggcacctttcgccgggcacggcgtcgaagcttcactccagctagctttctggaggaggacacaactgatttccccgatgagctggacacatccttctttgcccgggaaggtatcctccatgaagagctgtccacatacccggatgaagttttcgagtccccatcggaggcagcgctaaaggactgggagaaggcaccggagcaggcggacctcaccggcggggccctggaccgcagcgagcttgagcgcagccacctgatgctgcccttggagcgaggctggcggaagcagaaggagggcgccgcagccccgcagcccaaggtgcggctccgacaggaggtggtgagcaccgcggggccgcgacggggccagcgtatcgcggtgccggtgcgcaagctcttcgcccgggagaagcggccgtatgggctgggcatggtgggacggctcaccaaccgcacctaccgcaagcgcatcgacagcttcgtcaagcgccagatcgaggacatggacgaccacaggcccttcttcacctactggcttaccttcgtgcactcgctcgtcaccatcctagccgtgtgcatctatggcatcgcgcccgtgggcttctcgcagcatgagacggtggactcggtgctgcggaaccgcggggtctacgagaacgtcaagtacgtgcagcaggagaacttctggatcgggcccagctcggaggccctcatccacctgggcgccaagttttcgccctgcatgcgccaggacccgcaggtgcacagcttcattcgctcggcgcgcgagcgcgagaagcactccgcctgctgcgtgcgcaacgacaggtcgggctgcgtgcagacctcggaggaggagtgctcgtccacgctggcagtgtgggtgaagtggcccatccatcccagcgccccagagcttgcgggccacaagagacagtttggctctgtctgccaccaggatcccagggtgtgtgatgagccctcctccgaagaccctcatgagtggccagaagacatcaccaagtggccgatctgcaccaaaaacagcgctgggaaccacaccaaccatccccacatggactgtgtcatcacaggacggccctgctgcattggcaccaagggcaggtgtgagatcacctcccgggagtactgtgacttcatgaggggctacttccatgaggaggccacgctctgctctcaggtgcactgcatggatgatgtgtgtgggctcctgccttttctcaaccccgaggtgcctgaccagttctaccgcctgtggctatccctcttcctgcacgccgggatcttgcactgcctggtgtccatctgcttccagatgactgtcctgcgggacctggagaagctggcaggctggcaccgcatagccatcatctacctgctgagtggtgtcaccggcaacctggccagtgccatcttcctgccataccgagcagaggtgggtcctgctggctcccagttcggcatcctggcctgcctcttcgtggagctcttccagagctggcagatcctggcgcggccctggcgtgccttcttcaagctgctggctgtggtgctcttcctcttcacctttgggctgctgccgtggattgacaactttgcccacatctcggggttcatcagtggcctcttcctctccttcgccttcttgccctacatcagctttggcaagttcgacctgtaccggaaacgctgccagatcatcatctttcaggtggtcttcctgggcctcctggctggcctggtggtcctcttctacgtctatcctgtccgctgtgagtggtgtgagttcctcacctgcatccccttcactgacaagttctgtgagaagtacgaactggacgctcagctccactgagctggctgcgggctccagcggccgtgtgctccagcaggccagagccagacacgacctccctgagcctcacaggcttacaggagtcacctgctccatgtggggactggcctgtttcctgaacacagacctctttcttgtgccttgttcacttctgttgaacccctcgtactgccgggcatttattatactacttcctgtcataaccttctaacttgtttcttgacgaccacctcatgtggccaataaatggactgggagcgttttagctgccattaacttgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - protein, 855 aa, Homo sapiens
MSEARRDSTSSLQRKKPPWLKLDIPSAVPLTAEEPSFLQPLRRQAFLRSVSMPAETAHISSPHHELRRPVLQRQTSITQTIRRGTADWFGVSKDSDSTQKWQRKSIRHCSQRYGKLKPQVLRELDLPSQDNVSLTSTETPPPLYVGPCQLGMQKIIDPLARGRAFRVADDTAEGLSAPHTPVTPGAASLCSFSSSRSGFHRLPRRRKRESVAKMSFRAAAALMKGRSVRDGTFRRARRRSFTPASFLEEDTTDFPDELDTSFFAREGILHEELSTYPDEVFESPSEAALKDWEKAPEQADLTGGALDRSELERSHLMLPLERGWRKQKEGAAAPQPKVRLRQEVVSTAGPRRGQRIAVPVRKLFAREKRPYGLGMVGRLTNRTYRKRIDSFVKRQIEDMDDHRPFFTYWLTFVHSLVTILAVCIYGIAPVGFSQHETVDSVLRNRGVYENVKYVQQENFWIGPSSEALIHLGAKFSPCMRQDPQVHSFIRSAREREKHSACCVRNDRSGCVQTSEEECSSTLAVWVKWPIHPSAPELAGHKRQFGSVCHQDPRVCDEPSSEDPHEWPEDITKWPICTKNSAGNHTNHPHMDCVITGRPCCIGTKGRCEITSREYCDFMRGYFHEEATLCSQVHCMDDVCGLLPFLNPEVPDQFYRLWLSLFLHAGILHCLVSICFQMTVLRDLEKLAGWHRIAIIYLLSGVTGNLASAIFLPYRAEVGPAGSQFGILACLFVELFQSWQILARPWRAFFKLLAVVLFLFTFGLLPWIDNFAHISGFISGLFLSFAFLPYISFGKFDLYRKRCQIIIFQVVFLGLLAGLVVLFYVYPVRCEWCEFLTCIPFTDKFCEKYELDAQLH

SEQ ID NO: 3 - DNA, 3031 bp, Homo sapiens
ccgcacccccggctcggggctgtgagcggctcggggccgggggtgggcggcggtgcggcgggcggccgacgctcctcttcggcggcggcggcggcggcggcccatgcgtggggcggcgcggctggggcggccgggccggagttgcctcccggggcccgcgctgagggccgccgccgcgcccgccctgctgcttgctcgttgcgctgttgccgctgctgccggcctgcgtgccgccgcccgtccgcggcccccggagctgcagtcggcttccgcggggcccagcgtgagtctctacctgagcgaggacgaggtgcgccggctgatcggtcttgatgcagaactttattatgtgagaaatgaccttattagtcactacgctctatcctttaatctgttagtacccagtgagacaaatttcctgcacttcacctggcatgcgaagtccaaggttgaatataagctgggattccaagtggacaatgttttggcaatggatatgccccaggtcaacatttctgttcagggggaagttccacgcactttatcagtgtttcgggtagagctttcctgtactggcaaagtagattctgaagttatgatactaatgcagctcaacttgacagtaaattcttcaaaaaattttaccgtcttaaattttaaacgaaggaaaatgtgctacaaaaaacttgaagaagtaaaaacttcagccttggacaaaaacactagcagaactatttatgatcctgtacatgcagctccaaccacttctacgcgtgtgttttatattagtgtaggggtttgttgtgcagtaatatttctcgtagcaataatattagctgttttgcaccttcataatatgaaaaggattgaactggatgacagcattagtgccagcagtagttcccaagggctgtctcagccatccacccagacgactcagtatctgagagcagacacgcccaacaatgcaactcctatcaccagttatcctaccttgcggatagagaagaacgacttgagaagtgtcactcttttggaggccaaaggcaaggtgaaggatatagcaatatccagagagaggataactctaaaagatgtactccaagaaggtacttttgggcgtattttccatgggattttaatagatgaaaaagatccaaataaagaaaaacaagcatttgtcaaaacagttaaagatcaagcttctgaaattcaggtgacaatgatgctcactgaaagttgtaagctgcgaggtcttcatcacagaaatcttcttcctattactcatgtgtgtatagaagaaggagaaaagcccatggtgatattgccttacatgaattgggggaatcttaaattgtttttacgacagtgcaagttagtagaggccaataatccacaggcaatttctcagcaagacctggtacacatggctattcagattgcctgtggaatgagctacctggccagaagggaagtcatccacaaagacctggctgccaggaactgtgtcattgatgacacacttcaagttaagatcacagacaatgccctctccagagacttgttccccatggactatcactgtctgggggacaatgaaaacaggccagttcgttggatggctcttgaaagtctggttaataacgagttctctagcgctagtgatgtgtgggcctttggagtgacgctgtgggaactcatgactctgggccagactccctacgtggacattgaccccttcgagatggccgcatacctgaaagatggttaccgaatagcccagccaatcaactgtcctgatgaattatttgctgtgatggcctgttgctgggccttagatccagaggagaggcccaagtttcagcagctggtacagtgcctaacagagtttcatgcagccctgggggcctacgtctgactcctctccaatcccacaccatcaggaagaaggtgcctgtcggggctcacttgaagcctgtcagggatgctttgtatctaacacaacgccaacagaagcacatttgtcttccagaacaccgtgccttagaaatgctttagaatctgaactttttaagacagacttaataatgtggcatattttctagatatcacttttattaggttgaactgaaagggtttttgtaaattttttggccaaaattttttaaaacatacttactttggactaggggtacattcttacaaaataaataaacagtttttaaaattgtttagacacagatatttggaattagctatcttagggccaactgctttttatttttttacttcatcaaggtgatgtaagtgactttgtttagacacagatatttggaattagcctatcttagtgccaactgctttttatttttttccttcatcaaggtgatgtaagtgactcacctttaaagtttttttagtgttattttttatcactactctgggaaatggtttgtcttcaagatgcaatacttttcttagataaggaaaaacagcataaaaagatacctggtctgccttgtacaagaaaaccgaatattagaggaagaaaatttaaagaaaagctagagaaaaaaaaaattttttaaaaaatacttattagaagcaaactgcccttgcatggaaaactgtttatttttttcagtgaaaaaggaattctgctttcgtgtttttgggaaagcaggaactgagttcattacatctttaatttggcagaaattagcctttctgtgaaccagatgtggtttggggcagatctgtagtaaacaatggtgattttatttatttttactctctggaaaaggagataatacaattccagaaagtgaactcatatttctaaggtaagatcccttttatgcacctagaatatgctatgcacagagcgggtgcttgagttgttgtcgttttttgtttgttttttaaatgtaaactggtaaattttgtgcttatcttcaaggctggcttaagtataaaattgttttttaaacacttgaaaaattaaaggatttgttttatattaaaaaaaaaa

SEQ ID NO: 4 - protein, 604 aa, Homo sapiens
MRGAARLGRPGRSCLPGPALRAAAAPALLLARCAVAAAAGLRAAARPRPPELQSASAGPSVSLYLSEDEVRRLIGLDAELYYVRNDLISHYALSFNLLVPSETNFLHFTWHAKSKVEYKLGFQVDNVLAMDMPQVNISVQGEVPRTLSVFRVELSCTGKVDSEVMILMQLNLTVNSSKNFTVLNFKRRKMCYKKLEEVKTSALDKNTSRTIYDPVHAAPTTSTRVFYISVGVCCAVIFLVAIILAVLHLHNMKRIELDDSISASSSSQGLSQPSTQTTQYLRADTPNNATPITSYPTLRIEKNDLRSVTLLEAKGKVKDIAISRERITLKDVLQEGTFGRIFHGILIDEKDPNKEKQAFVKTVKDQASEIQVTMMLTESCKLRGLHHRNLLPITHVCIEEGEKPMVILPYMNWGNLKLFLRQCKLVEANNPQAISQQDLVHMAIQIACGMSYLARREVIHKDLAARNCVIDDTLQVKITDNALSRDLFPMDYHCLGDNENRPVRWMALESLVNNEFSSASDVWAFGVTLWELMTLGQTPYVDIDPFEMAAYLKDGYRIAQPINCPDELFAVMACCWALDPEERPKFQQLVQCLTEFHAALGAYV

SEQ ID NO: 5 - DNA, 1638 bp, Homo sapiens
cgggccctgcgggcgcggggctgaaggcggaaccacgacgggcagagagcacggagccgggaagcccctgggcgcccgtcggagggctatggagcagcggccgcggggctgcgcggcggtggcggcggcgctcctcctggtgctgctgggggcccgggcccagggcggcactcgtagccccaggtgtgactgtgccggtgacttccacaagaagattggtctgttttgttgcagaggctgcccagcggggcactacctgaaggccccttgcacggagccctgcggcaactccacctgccttgtgtgtccccaagacaccttcttggcctgggagaaccaccataattctgaatgtgcccgctgccaggcctgtgatgagcaggcctcccaggtggcgctggagaactgttcagcagtggccgacacccgctgtggctgtaagccaggctggtttgtggagtgccaggtcagccaatgtgtcagcagttcacccttctactgccaaccatgcctagactgcggggccctgcaccgccacacacggctactctgttcccgcagagatactgactgtgggacctgcctgcctggcttctatgaacatggcgatggctgcgtgtcctgccccacgagcaccctggggagctgtccagagcgctgtgccgctgtctgtggctggaggcagatgttctgggtccaggtgctcctggctggccttgtggtccccctcctgcttggggccaccctgacctacacataccgccactgctggcctcacaagcccctggttactgcagatgaagctgggatggaggctctgaccccaccaccggccacccatctgtcacccttggacagcgcccacacccttctagcacctcctgacagcagtgagaagatctgcaccgtccagttggtgggtaacagctggacccctggctaccccgagacccaggaggcgctctgcccgcaggtgacatggtcctgggaccagttgcccagcagagctcttggccccgctgctgcgcccacactctcgccagagtccccagccggctcgccagccatgatgctgcagccgggcccgcagctctacgacgtgatggacgcggtcccagcgcggcgctggaaggagttcgtgcgcacgctggggctgcgcgaggcagagatcgaagccgtggaggtggagatcggccgcttccgagaccagcagtacgagatgctcaagcgctggcgccagcagcagcccgcgggcctcggagccgtttacgcggccctggagcgcatggggctggacggctgcgtggaagacttgcgcagccgcctgcagcgcggcccgtgacacggcgcccacttgccacctaggcgctctggtggcccttgcagaagccctaagtacggttacttatgcgtgtagacattttatgtcacttattaagccgctggcacggccctgcgtagcagcaccagccggccccacccctgctcgcccctatcgctccagccaaggcgaagaagcacgaacgaatgtcgagagggggtgaagacatttctcaacttctcggccggagtttggctgagatcgcggtattaaatctgtgaaagaaaacaaaacaaaacaaaaaaaaaaaaaaaaaa

SEQ ID NO: 6 - protein, 1148 aa, Homo sapiens
METGLGLNARGPRARGGLYCYSALAALAVALALAALAALALELELEVALLELEGLYALAARGALAGLNGLYGLYTHRARGSERPRARGCYSASPCYSALAGLYASPPHEHISLYSLYSILEGLYLEPHECYSCYSARGGLYCYSPRALAGLYHISTYRLELYSALAPRCYSTHRGLPRCYSGLYASNSERTHRCYSLEVALCYSPRGLNASPTHRPHELEALATRPGLASNHISHISASNSERGLCYSALAARGCYSGLNALACYSASPGLGLNALASERGLNVALALALEGLASNCYSSERALAVALALAASPTHRARGCYSGLYCYSLYSPRGLYTRPPHEVALGLCYSGLNVALSERGLNCYSVALSERSERSERPRPHETYRCYSGLNPRCYSLEASPCYSGLYALALEHISARGHISTHRARGLELECYSSERARGARGASPTHRASPCYSGLYTHRCYSLEPRGLYPHETYRGLHISGLYASPGLYCYSVALSERCYSPRTHRSERTHRLEGLYSERCYSPRGLARGCYSALAALAVALCYSGLYTRPARGGLNMETPHETRPVALGLNVALLELEALAGLYLEVALVALPRLELELEGLYALATHRLETHRTYRTHRTYRARGHISCYSTRPPRHISLYSPRLEVALTHRALAASPGLALAGLYMETGLALALETHRPRPRPRALATHRHISLESERPRLEASPSERALAHISTHRLELEALAPRPRASPSERSERGLLYSILECYSTHRVALGLNLEVALGLYASNSERTRPTHRPRGLYTYRPRGLTHRGLNGLALALECYSPRGLNVALTHRTRPSERTRPASPGLNLEPRSERARGALALEGLYPRALAALAALAPRTHRLESERPRGLSERPRALAGLYSERPRALAMETMETLEGLNPRGLYPRGLNLETYRASPVALMETASPALAVALPRALAARGARGTRPLYSGLPHEVALARGTHRLEGLYLEARGGLALAGLILEGLALAVALGLVALGLILEGLYARGPHEARGASPGLNGLNTYRGLMETLELYSARGTRPARGGLNGLNGLNPRALAGLYLEGLYALAVALTYRALAALALEGLARGMETGLYLEASPGLYCYSVALGLASPLEARGSERARGLEGLNARGGLYPR

SEQ ID NO: 7 - DNA, 7259 bp, Homo sapiens
  misc_feature (48)..(48): n = A,T,C or G
ccacgcgtccgcagccccgtcgccggcggaggcgggcgcgggcgcgtnccctgtggccagtcacccggaggagttggtcgcacaattatgaaagactcggcttctgctgctagcgccggagctgagttagttctgagaaggtttccctgggcgttccttgtccggcggcctctgctgccgcctccggagacgcttcccgatagatggctacaggccgcggaggaggaggaggtggagttgctgcccttccggagtccgccccgtgaggagaatgtcccagaaatcctggatagaaagcactttgaccaagagggaatgtgtatatattataccaagttccaaggaccctcacagatgccttccaggatgtcaaatttgtcagcaactcgtcaggtgtttttgtggtcgcttggtcaagcaacatgcttgttttactgcaagtcttgccatgaaatactcagatgtgaaattgggtgaccattttaatcaggcaatagaagaatggtctgtggaaaagcatacagaacagagcccaacggatgcttatggagtcataaattttcaagggggttctcattcctacagagctaagtatgtgaggctatcatatgacaccaaacctgaagtcattctgcaacttctgcttaaagaatggcaaatggagttacccaaacttgttatctctgtacatgggggcatgcagaaatttgagcttcacccacgaatcaagcagttgcttggaaaaggtcttattaaagctgcagttacaactggagcctggattttaactggaggagtaaacacaggtgtggcaaaacatgttggagatgccctcaaagaacatgcttccagatcatctcgaaagatttgcactatcggaatagctccatggggagtgattgaaaacagaaatgatcttgttgggagagatgtggttgctccttatcaaaccttattgaaccccctgagcaaattgaatgttttgaataatctgcattcccatttcatattggtggatgatggcactgttggaaagtatggggcggaagtcagactgagaagagaacttgaaaaaactattaatcagcaaagaattcatgctaggattggccagggtgtccctgtggtggcacttatatttgagggtgggccaaatgttatcctcacagttcttgaataccttcaggaaagcccccctgttccagtagttgtgtgtgaaggaacaggcagagctgcagatctgctagcgtatattcataaacaaacagaagaaggagggaatcttcctgatgcagcagagcccgatattatttccactatcaaaaaaacatttaactttggccagaatgaagcacttcatttatttcaaacactgatggagtgcatgaaaagaaaggagcttatcactgttttccatattgggtcagatgaacatcaagatatagatgtagcaatacttactgcactgctaaaaggtactaatgcatctgcatttgaccagcttatccttacattggcatgggatagagttgacattgccaaaaatcatgtatttgtttatggacagcagtggctggttggatccttggaacaagctatgcttgatgctcttgtaatggatagagttgcatttgtaaaacttcttattgaaaatggagtaagcatgcataaattccttaccattccgagactggaagaactttacaacactaaacaaggtccaactaatccaatgctgtttcatcttgttcgagacgtcaaacagggaaatcttcctccaggatataagatcactctgattgatataggacttgttattgaatatctcatgggaggaacctacagatgcacctatactaggaaacgttttcgattaatatataatagtcttggtggaaataatcggaggtctggccgaaatacctccagcagcactcctcagttgcgaaagagtcatgaatcttttggcaatagggcagataaaaaggaaaaaatgaggcataaccatttcattaagacagcacagccctaccgaccaaagattgatacagttatggaagaaggaaagaagaaaagaaccaaagatgaaattgtagacattgatgatccagaaaccaagcgctttccttatccacttaatgaacttttaatttgggcttgccttatgaagaggcaggtcatggcccgttttttatggcaacatggtgaagaatcaatggctaaagcattagttgcctgtaagatctatcgttcaatggcatatgaagcaaagcagagtgacctggtagatgatacttcagaagaactaaaacagtattccaatgattttggtcagttggccgttgaattattagaacagtccttcagacaagatgaaaccatggctatgaaattgctcacttatgaactgaagaactggagtaattcaacctgccttaagttagcagtttcttcaagacttagaccttttgtagctcacacctgtacacaaatgttgttatctgatatgtggatgggaaggctgaatatgaggaaaaattcctggtacaaggtcatactaagcattttagttccacctgccatattgctgttagagtataaaactaaggctgaaatgtcccatatcccacaatctcaagatgctcatcagatgacaatggatgacagcgaaaacaactttcagaacataacagaagagatccccatggaagtgtttaaagaagtacggattttggatagtaatgaaggaaagaatgagatggagatacaaatgaaatcaaaaaagcttccaattacgcgaaagttttatgccttttatcatgcaccaattgtaaaattctggtttaacacgttggcatatttaggatttctgatgctttatacatttgtggttcttgtacaaatggaacagttaccttcagttcaagaatggattgttattgcttatatttttacttatgccattgagaaagtccgtgagatctttatgtctgaagctgggaaagtaaaccagaagattaaagtatggtttagtgattacttcaacatcagtgatacaattgccataatttctttcttcattggatttggactaagatttggagcaaaatggaactttgcaaatgcatatgataatcatgtttttgtggctggaagattaatttactgtcttaacataatattttggtatgtgcgtttgctagattttctagctgtaaatcaacaggcaggaccttatgtaatgatgattggaaaaatggtggccaatatgttctacattgtagtgattatggctcttgtattacttagttttggtgttcccagaaaggcaatactttatcctcatgaagcaccatcttggactcttgctaaagatatagtttttcacccatactggatgatttttggtgaagtttatgcatacgaaattgatgtgtgtgcaaatgattctgttatccctcaaatctgtggtcctgggacgtggttgactccatttcttcaagcagtctacctctttgtacagtatatcattatggttaatcttcttattgcatttttcaacaatgtgtatttacaagtgaaggcaatttccaatattgtatggaagtaccagcgttatcattttattatggcttatcatgagaaaccagttctgcctcctccacttatcattcttagccatatagtttctctgttttgctgcatatgtaagagaagaaagaaagataagacttccgatggaccaaaacttttcttaacagaagaagatcaaaagaaacttcatgattttgaagagcagtgtgttgaaatgtatttcaatgaaaaagatgacaaatttcattctgggagtgaagagagaattcgtgtcacttttgaaagagtggaacagatgtgcattcagattaaagaagttggagatcgtgtcaactacataaaaagatcattacaatcattagattctcaaattggccatttgcaagatctttcagccctgacggtagatacattaaaaacactcactgcccagaaagcgtcggaagctagcaaagttcataatgaaatcacacgagaactgagcatttccaaacacttggctcaaaaccttattgatgatggtcctgtaagaccttctgtatggaaaaagcatggtgttgtaaatacacttagctcctctcttcctcaaggtgatcttgaaagtaataatccttttcattgtaatattttaatgaaagatgacaaagatccccagtgtaatatatttggtcaagacttacctgcagtaccccagagaaaagaatttaattttccagaggctggttcctcttctggtgccttattcccaagtgctgtttcccctccagaactgcgacagagactacatggggtagaactcttaaaaatatttaataaaaatcaaaaattaggcagttcatctactagcataccacatctgtcatccccaccaaccaaattttttgttagtacaccatctcagccaagttgcaaaagccacttggaaactggaaccaaagatcaagaaactgtttgctctaaagctacagaaggagataatacagaatttggagcatttgtaggacacagagatagcatggatttacagaggtttaaagaaacatcaaacaagataaaaatactatccaataacaatacttctgaaaacactttgaaacgagtgagttctcttgctggatttactgactgtcacagaacttccattcctgttcattcaaaacaagcagaaaaaatcagtagaaggccatctaccgaagacactcatgaagtagattccaaagcagctttaataccggattggttacaagatagaccatcaaacagagaaatgccatctgaagaaggaacattaaatggtctcacttctccatttaagccagctatggatacaaattactattattcagctgtggaaagaaataacttgatgaggttatcacagagcattccatttacacctgtgcctccaagaggggagcctgtcacagtgtatcgtttggaagagagttcacccaacatactaaataacagcatgtcttcttggtcacaactaggcctctgtgccaaaatagagtttttaagcaaagaggagatgggaggaggtttacgaagagctgtcaaagtacagtgtacctggtcagaacatgatatcctcaaatcagggcatctttatattatcaaatcttttcttccagaggtggttaatacatggtcaagtatttataaagaagatacagttctgcatctctgtctgagagaaattcaacaacagagagcagcacaaaagcttacgtttgcctttaatcaaatgaaacccaaatccataccatattctccaaggttccttgaagttttcctgctgtattgccattcagcaggacagtggtttgctgtggaagaatgtatgactggagaatttagaaaatacaacaataataatggagatgagattattccaactaatactctggaagagatcatgctagcctttagccactggacttacgaatatacaagaggggagttactggtacttgatttgcaaggtgttggtgaaaatttgactgacccatctgtgataaaagcagaagaaaagagatcctgtgatatggtttttggcccagcaaatctaggagaagatgcaattaaaaacttcagagcaaaacatcactgtaattcttgctgtagaaagcttaaacttccagatctgaagaggaatgattatacgcctgataaaattatatttcctcaggatgagccttcagatttgaatcttcagcctggaaattccaccaaagaatcagaatcaactaattctgttcgtctgatgttataatattaatattactgaatcattggttttgcctgcacctcacagaaatgttactgtgtcacttttccctcgggaggaaattgtttggtaatatagaaaggtgtatgcaagttgaatttgctgactccagcacagttaaaaggtcaatattcttttgacctgattaatcagtcagaaagtccctataggatagagctggcagctgagaaattttaaaggtaattgataattagtatttgtaactttttaaagggctctttgtatagcagaggatctcatttgactttgttttgatgagggtgatgccctctcttatgtggtacaataccattaaccaaaggtaggtgtccatgcagattttattggcagctgttttattgccattcaactagggaaatgaagaaatcacgcagccttttggttaaatggcagtcaaaattttcctcagtgtatttagtgtgttcagtgatgatatcactggttcccaactagatgcttgttggccacgggaagggaaatgacttgttctaattctaggttcacagaggtatgagaagcctgaactgaagaccattttcaagagggacggtatttatgaatcagggttaggctccatatttaaagatagagccagttttttttttaaatagaacccaaattgtgtaaaaatgttaattgggttttttaaacattgttttatcaagtcactgttaagtagaagaaagccatggtaaactgatacataacctaaattataaaagcagaaacctaactcactcgtcaagggaagttaccttttgaggaaagttaaagtacttttttccctatctgtatctatagcaacaacccagaacttacaaacttctccaaagattttattgattgttatatcaaatcagaatgtaaacatgaactcttgcatatatttaaaattgtgttggaacatttgaacatgaatgctgtttgggtacttaagaaattrattcagtnggattatcattatgtganactggcagattgcagtgcanccttatgccaataaaatgtaatttaacagccccagatattgttgaatattcaacaataacaagaaaagcttttcatctaagttttatgctttaattttttttctttttttttctttttcttttgtttccttggtactaattttaatttttatttggaagggagcagtataaagcttatttgtatttagtagtgtatctcatagatacagacaaggcaagagatgataagctgtttaaatagtgtttaatattgattgggggtggggagaaagaaaaagtgtattacttaaagatactatatacgttttgtatatcattaaatctttaaaagaaatnnaataaatttattgtttncaaaaaaaaaa

SEQ ID NO: 8 - protein, 1865 aa, Homo sapiens
MSQKSWIESTLTKRECVYIIPSSKDPHRCLPGCQICQQLVRCFCGRLVKQHACFTASLAMKYSDVKLGDHFNQAIEEWSVEKHTEQSPTDAYGVINFQGGSHSYRAKYVRLSYDTKPEVILQLLLKEWQMELPKLVISVHGGMQKFELHPRIKQLLGKGLIKAAVTTGAWILTGGVNTGVAKHVGDALKEHASRSSRKICTIGIAPWGVIENRNDLVGRDVVAPYQTLLNPLSKLNVLNNLHSHFILVDDGTVGKYGAEVRLRRELEKTINQQRIHARIGQGVPVVALIFEGGPNVILTVLEYLQESPPVPVVVCEGTGRAADLLAYIHKQTEEGGNLPDAAEPDIISTIKKTFNFGQNEALHLFQTLMECMKRKELITVFHIGSDEHQDIDVAILTALLKGTNASAFDQLILTLAWDRVDIAKNHVFVYGQQWLVGSLEQAMLDALVMDRVAFVKLLIENGVSMHKFLTIPRLEELYNTKQGPTNPMLFHLVRDVKQGNLPPGYKITLIDIGLVIEYLMGGTYRCTYTRKRFRLIYNSLGGNNRRSGRNTSSSTPQLRKSHESFGNRADKKEKMRHNHFIKTAQPYRPKIDTVMEEGKKKRTKDEIVDIDDPETKRFPYPLNELLIWACLMKRQVMARFLWQHGEESMAKALVACKIYRSMAYEAKQSDLVDDTSEELKQYSNDFGQLAVELLEQSFRQDETMAMKLLTYELKNWSNSTCLKLAVSSRLRPFVAHTCTQMLLSDMWMGRLNMRKNSWYKVILSILVPPAILLLEYKTKAEMSHIPQSQDAHQMTMDDSENNFQNITEEIPMEVFKEVRILDSNEGKNEMEIQMKSKKLPITRKFYAFYHAPIVKFWFNTLAYLGFLMLYTFVVLVQMEQLPSVQEWIVIAYIFTYAIEKVREIFMSEAGKVNQKIKVWFSDYFNISDTIAIISFFIGFGLRFGAKWNFANAYDNHVFVAGRLIYCLNIIFWYVRLLDFLAVNQQAGPYVMMIGKMVANMFYIVVIMALVLLSFGVPRKAILYPHEAPSWTLAKDIVFHPYWMIFGEVYAYEIDVCANDSVIPQICGPGTWLTPFLQAVYLFVQYIIMVNLLIAFFNNVYLQVKAISNIVWKYQRYHFIMAYHEKPVLPPPLIILSHIVSLFCCICKRRKKDKTSDGPKLFLTEEDQKKLHDFEEQCVEMYFNEKDDKFHSGSEERIRVTFERVEQMCIQIKEVGDRVNYIKRSLQSLDSQIGHLQDLSALTVDTLKTLTAQKASEASKVHNEITRELSISKHLAQNLIDDGPVRPSVWKKHGVVNTLSSSLPQGDLESNNPFHCNILMKDDKDPQCNIFGQDLPAVPQRKEFNFPEAGSSSGALFPSAVSPPELRQRLHGVELLKIFNKNQKLGSSSTSIPHLSSPPTKFFVSTPSQPSCKSHLETGTKDQETVCSKATEGDNTEFGAFVGHRDSMDLQRFKETSNKIKILSNNNTSENTLKRVSSLAGFTDCHRTSIPVHSKQAEKISRRPSTEDTHEVDSKAALIPDWLQDRPSNREMPSEEGTLNGLTSPFKPAMDTNYYYSAVERNNLMRLSQSIPFTPVPPRGEPVTVYRLEESSPNILNNSMSSWSQLGLCAKIEFLSKEEMGGGLRRAVKVQCTWSEHDILKSGHLYIIKSFLPEVVNTWSSIYKEDTVLHLCLREIQQQRAAQKLTFAFNQMKPKSIPYSPRFLEVFLLYCHSAGQWFAVEECMTGEFRKYNNNNGDEIIPTNTLEEIMLAFSHWTYEYTRGELLVLDLQGVGENLTDPSVIKAEEKRSCDMVFGPANLGEDAIKNFRAKHHCNSCCRKLKLPDLKRNDYTPDKIIFPQDEPSDLNLQPGNSTKESESTNSVRLML

SEQ ID NO: 9 - DNA, 3770 bp, Homo sapiens
gagactggggccagggagacagccctgggggagaggcgcccgaaccaggccgcgggagcatgggggcccggagcggagctcggggcgcgctgctgctggcactgctgctctgctgggacccgaggctgagccaagcaggcactgattctggcagcgaggtgctccctgactccttcccgtcagcgccagcagagccgctgccctacttcctgcaggagccacaggacgcctacattgtgaagaacaagcctgtggagctccgctgccgcgccttccccgccacacagatctacttcaagtgcaacggcgagtgggtcagccagaacgaccacgtcacacaggaaggcctggatgaggccaccggcctgcgggtgcgcgaggtgcagatcgaggtgtcgcggcagcaggtggaggagctctttgggctggaggattactggtgccagtgcgtggcctggagctccgcaggcaccaccaagagtcgccgagcctacgtccgcatcgcctacctgcgcaagaacttcgatcaggagcctctgggcaaggaggtgcccctggaccatgaggttctcctgcagtgccgcccgccggagggggtgcctgtggccgaggtggaatggctcaagaatgaggatgtcatcgaccccacccaggacaccaacttcctgctcaccatcgaccacaacctcatcatccgccaggcccgcctgtcggacactgccaactatacctgcgtggccaagaacatcgtggccaaacgccggagcaccactgccaccgtcatcgtctacgtgaatggcggctggtccagctgggcagagtggtcaccctgctccaaccgctgtggccgaggctggcagaagcgcacccggacctgcaccaaccccgctccactcaacggaggggccttctgcgagggccaggcattccagaagaccgcctgcaccaccatctgcccagtcgatggggcgtggacggagtggagcaagtggtcagcctgcagcactgagtgtgcccactggcgtagccgcgagtgcatggcgcccccaccccagaacggaggccgtgactgcagcgggacgctgctcgactctaagaactgcacagatgggctgtgcatgcaaatgctggaggcctcaggggatgcggcgctgtatgcggggctcgtggtggccatcttcgtggtcgtggcaatcctcatggcggtgggggtggtggtgtaccgccgcaactgccgtgacttcgacacagacatcactgactcatctgctgccctgactggtggtttccaccccgtcaactttaagacggcaaggcccagcaacccgcagctcctacacccctctgtgcctcctgacctgacagccagcgccggcatctaccgcggacccgtgtatgccctgcaggactccaccgacaaaatccccatgaccaactctcctctgctggaccccttacccagccttaaggtcaaggtctacagctccagcaccacgggctctgggccaggcctggcagatggggctgacctgctgggggtcttgccgcctggcacataccctagcgatttcgcccgggacacccacttcctgcacctgcgcagcgccagcctcggttcccagcagctcttgggcctgccccgagacccagggagcagcgtcagcggcacctttggctgcctgggtgggaggctcagcatccccggcacaggggtcagcttgctggtgcccaatggagccattccccagggcaagttctacgagatgtatctactcatcaacaaggcagaaagtaccctcccgctttcagaagggacccagacagtattgagcccctcggtgacctgtggacccacaggcctcctgctgtgccgccccgtcatcctcaccatgccccactgtgccgaagtcagtgcccgtgactggatctttcagctcaagacccaggcccaccagggccactgggaggaggtggtgaccctggatgaggagaccctgaacacaccctgctactgccagctggagcccagggcctgtcacatcctgctggaccagctgggcacctacgtgttcacgggcgagtcctattcccgctcagcagtcaagcggctccagctggccgtcttcgcccccgccctctgcacctccctggagtacagcctccgggtctactgcctggaggacacgcctgtagcactgaaggaggtgctggagctggagcggactctgggcggatacttggtggaggagccgaaaccgctaatgttcaaggacagttaccacaacctgcgcctctccctccatgacctcccccatgcccattggaggagcaagctgctggccaaataccaggagatccccttctatcacatttggagtggcagccagaaggccctccactgcactttcaccctggagaggcacagcttggcctccacagagctcacctgcaagatctgcgtgcggcaagtggaaggggagggccagatattccagctgcataccactctggcagagacacctgctggctccctggacactctctgctctgcccctggcagcactgtcaccacccagctgggaccttatgccttcaagatcccactgtccatccgccagaagatatgcaacagcctagatgcccccaactcacggggcaatgactggcggatgttagcacagaagctctctatggaccggtacctgaattactttgccaccaaagcgagccccacgggtgtgatcctggacctctgggaagctctgcagcaggacgatggggacctcaacagcctggcgagtgccttggaggagatgggcaagagtgagatgctggtggctgtggccaccgacggggactgctgagcctcctgggacagcgggctggcagggactggcaggaggcaggtgcagggaggcctggggcagcctcctgatggggatgtttggcctctgcttcctcccagttcacagccagagttgcctctcctcctcctcttccccaacccccagaccatgaccagccttagaaaatccatgtactctgttgttagagggcccagagttccttctccacccccgctctctctctcttggcctgagatctctgtgcaggaaccaagatggggctgaagcctctggaggcagttggttgggggcgggcaggcaggaagcccttccttcacccccccacccttagcccggaacttctgggttccatgggttttaagttccgttcttcgttttcttcctccgtattgattttctccttttttccctaagccccctttgcttccacccctttttctctttgagagtcaagtacaatttaaaacaaacttgcttttttcttttccaaaacaaaaaggcaaggaaaaaaagaaagcttcaaaccgtttgtaaggttaaagaaaaaaaaaaccctccacccccccaccctcagcccggcaacttctgggttccatgggttttagttccgttctcgttttcttcctccgttattgatttctcctttctccctaagcccccttctgcttccacgcccttttcctctttgaagagtcaagtacaattcagacaaactgctttctcctgtccaaaagcaaaaaggcaaaggaaagaaagaaagcttcaaaccgctagtaaggctcaaagaagaagaaaaacaccaaaaccacaagggaaaagaaaaacccagtttcttaggaaacgcaaacgatttattatccagattatttggataagtcctttttaagaaaaaaaaaaaaaaaa

SEQ ID NO: 10 - protein, 934 aa, Homo sapiens
MGARSGARGALLLALLLCWDPRLSQAGTDSGSEVLPDSFPSAPAEPLPYFLQEPQDAYIVKNKPVELRCRAFPATQIYFKCNGEWVSQNDHVTQEGLDEATGLRVREVQIEVSRQQVEELFGLEDYWCQCVAWSSAGTTKSRRAYVRIAYLRKNFDQEPLGKEVPLDHEVLLQCRPPEGVPVAEVEWLKNEDVIDPTQDTNFLLTIDHNLIIRQARLSDTANYTCVAKNIVAKRRSTTATVIVYVNGGWSSWAEWSPCSNRCGRGWQKRTRTCTNPAPLNGGAFCEGQAFQKTACTTICPVDGAWTEWSKWSACSTECAHWRSRECMAPPPQNGGRDCSGTLLDSKNCTDGLCMQMLEASGDAALYAGLVVAIFVVVAILMAVGVVVYRRNCRDFDTDITDSSAALTGGFHPVNFKTARPSNPQLLHPSVPPDLTASAGIYRGPVYALQDSTDKIPMTNSPLLDPLPSLKVKVYSSSTTGSGPGLADGADLLGVLPPGTYPSDFARDTHFLHLRSASLGSQQLLGLPRDPGSSVSGTFGCLGGRLSIPGTGVSLLVPNGAIPQGKFYEMYLLINKAESTLPLSEGTQTVLSPSVTCGPTGLLLCRPVILTMPHCAEVSARDWIFQLKTQAHQGHWEEVVTLDEETLNTPCYCQLEPRACHILLDQLGTYVFTGESYSRSAVKRLQLAVFAPALCTSLEYSLRVYCLEDTPVALKEVLELERTLGGYLVEEPKPLMFKDSYHNLRLSLHDLPHAHWRSKLLAKYQEIPFYHIWSGSQKALHCTFTLERHSLASTELTCKICVRQVEGEGQIFQLHTTLAETPAGSLDTLCSAPGSTVTTQLGPYAFKIPLSIRQKICNSLDAPNSRGNDWRMLAQKLSMDRYLNYFATKASPTGVILDLWEALQQDDGDLNSLASALEEMGKSEMLVAVATDGDC

SEQ ID NO: 11 - DNA, 723 bp, Homo sapiens
atgaggcgctgcagtctctgcgctttcgacgccgcccgggggcccaggcggctgatgcgtgtgggcctcgcgctgatcttggtgggccacgtgaacctgctgctgggggccgtgctgcatggcaccgtcctgcggcacgtggccaatccccgcggcgctgtcacgccggagtacaccgtagccaatgtcatctctgtcggctcggggctgctgagcgtttccgtgggacttgtgaccctcctggcgtccaggaaccttcttcgccctccactgcactgggtcctgctggcactagctctggtgaacctgctcttgtccgttgcctgctccctgggcctccttcttgctgtgtcactcactgtggccaacggtggccgccgccttattgctgactgccacccaggactgctggatcctctggtaccactggatgaggggccgggacatactgactgcccctttgaccccacaagaatctatgatacagccttggctctctggatcccttctttgctcatgtctgcaggggaggctgctctatctggttactgctgtgtggctgcactcactctacgtggagttgggccctgcaggaaggacggacttcaggggcagctagaggaaatgacagagctcgaatctcctaaatgtaaaaggcaggaaaatgagcagctactggatcaaaatcaagaaatccgggcatcacagagaagttgggtttag

SEQ ID NO: 12 - protein, 240 aa, Homo sapiens
MRRCSLCAFDAARGPRRLMRVGLALILVGHVNLLLGAVLHGTVLRHVANPRGAVTPEYTVANVISVGSGLLSVSVGLVTLLASRNLLRPPLHWVLLALALVNLLLSVACSLGLLLAVSLTVANGGRRLIADCHPGLLDPLVPLDEGPGHTDCPFDPTRIYDTALALWIPSLLMSAGEAALSGYCCVAALTLRGVGPCRKDGLQGQLEEMTELESPKCKRQENEQLLDQNQEIRASQRSWV

SEQ ID NO: 13 - DNA, 4943 bp, Homo sapiens
gtctgcgtaaccgcacgggaaacccgccaccacctccacctccctgccatcctcgacaagatgcctgcccccggcgccctcatcctccttgcggccgtctccgcctccggctgcctggcgtccccggcccaccccgatggattcgccctgggccgggctcctctggctcctccctacgctgtggtcctcatttcctgctccggcctgctggccttcatcttcctcctcctcacctgtctgtgctgcaaacggggcgatgtcggcttcaaggaatttgagaaccctgaaggggaggactgctccggggagtacactccccctgcggaggagacctcctcctcacagtcgctgcctgatgtctacattctcccgctggctgaggtctccctgccaatgcctgccccgcagccttcacactcagacatgaccacccccctgggccttagccggcagcacctgagctacctgcaggagattgggagtggctggtttgggaaggtgatcctgggagagattttctccgactacacccccgcccaggtggtggtgaaggagctccgagccagcgcggggcccctggagcaacgcaagttcatctcggaagcacagccgtacaggagcctgcagcaccccaatgtcctccagtgcctgggtctgtgcgtggagacgctgccgtttctgctgattatggagttctgtcaactgggggacctgaagcgttacctccgagcccagcggccccccgagggcctgtcccctgagctaccccctcgagacctgcggacgctgcagaggatgggcctggagatcgcccgcgggctggcgcacctgcattcccacaactacgtgcacagcgacctggccctgcgcaactgcctgctgacctctgacctgaccgtgcgcatcggagactacgggctggcccacagcaactacaaggaggactactacctgaccccagagcgcctgtggatcccactgcgctgggcggcgcccgagctcctcggggagctccacgggaccttcatggtggtggaccagagccgcgagagcaacatctggtccctgggggtgaccctgtgggagctgtttgagtttggggcccagccctaccgccacctgtcagacgaggaggtcctcgccttcgtggtccgccagcagcatgtgaagctggcccggccgaggctcaagctgccttacgcggactactggtatgacattcttcagtcctgctggcggccacctgcccagcgcccttcagcctctgatctccaattgcagctcacctacttgctctccgagcggcctccccggcccccaccgccgccacccccaccccgagacggtcccttcccctggccctggccccctgcacacagtgcgccccgcccggggaccctctcctcaccgttccccctactggatggcttccctggagccgaccccgacgatgtgctcacggtcaccgagagtagccgcggcctcaacctcgagtgcctgtgggagaaggcccggcgtggggccggccggggtgggggggcacctgcctggcagccggcgtcggcccccccggccccccacgccaacccctccaaccctttctacgaggcgctgtccacgcccagcgtgctgcctgtcatcagcgcccgcagcccctccgtgagcagcgagtactacatccgcttggaggagcacggctcccctcctgagcccctcttccccaacgactgggaccccctggacccaggagtgcccgcccctcaggccccccaggccccctccgaggtcccccagctggtgtccgagacctgggcctcccccctcttccctgcgccccggcccttcccagcccagtcctcagcgtcaggcagcttcctgctgagcggctgggaccccgagggccggggcgccggggagaccctggcgggagaccctgccgaggtcttgggggagcgggggaccgccccgtgggtggaagaagaagaggaggaggaggagggcagctccccaggggaagacagcagcagccttggaggtggcccaagccgccggggtcccctaccctgtcccctgtgcagccgcgagggggcctgctcctgcctgccactggagcggggggacgccgtagcaggctggggaggccaccctgctcttggctgcccccacccccccgaggacgactcctcgctgcgggcagagcggggctccctggccgacttgcccatggccccccccgcctcggccccccccgagtttctggaccccctcatgggggcggcggcgccccagtaccccgggcgggggccacctcccgctcccccccccccgccgccacctcctcgggcccccgcggacccggccgcgtcccccgaccccccttcggccgtggccagtcccggttcaggcctctcgtcgccgggccccaagccgggggacagcggctacgagaccgagacccctttttccccagagggagccttcccaggtgggggggcggccgaggaggaaggggtccctcggccgcgggctccccccgagccacccgacccaggagcgccccggccacctccagacccgggtccgctcccactcccggggccccgggagaagccgaccttcgtggttcaagtgagcacggaacagctgctgatgtccctgcgggaggatgtgacaaggaacctcctgggggagaagggggcgacagcccgggagacaggacccaggaaggcggggagaggccccgggaacagagagaaagtcccgggcctgaacagggacccgacagtcctgggcaacgggaaacaagccccaagcctgagcctcccagtgaacggggtgacagtgctggagaacggggaccagagagccccaggcatcgaggagaaggcggcggagaatggggccctggggtcccccgagagagaagagaaagtgctggagaatggggagctgacacccccaaggagggaggagaaagcgctggagaatggggagctgaggtccccagaggccggggagaaggtgctggtgaatgggggcctgacacccccaaagagcgaggacaaggtgtcagagaatgggggcctgagattccccaggaacacggagaggccaccagagactgggccttggagagccccagggccctgggagaagacgcccgagagttggggtccagcccccacgatcggggagccagccccagagacctctctggagagagcccctgcacccagcgcagtggtctcctcccggaacggcggggagacagcccctggcccccttggcccagcccccaagaacgggacgctggaacccgggaccgagaggagagcccccgagactgggggggcgccgagagccccaggggctgggaggctggacctcgggagtgggggccgagccccagtgggcacggggacggcccccggcggcggccccggaagcggcgtggacgcaaaggccggatgggtagacaacacgaggccgcagccaccgccgccaccgctgccaccgccaccggaggcacagccgaggaggctggagccagcgcccccgagagccaggccggaggtggcccccgagggagagcccggggccccagacagcagggccggcggagacacggcactcagcggagacggggacccccccaagcccgagaggaagggccccgagatgccacgactattcttggacttgggaccccctcaggggaacagcgagcagatcaaagccaggctctcccggctctcgctggcgctgccgccgctcacgctcacgccattcccggggccgggcccgcggcggcccccgtgggagggcgcggacgccggggcggctggcggggaggccggcggggcgggagcgccggggccggcggaggaggacggggaggacgaggacgaggacgaggaggaggacgaggaggcggcggcgccgggcgcggcggcggggccgcggggccccgggagggcgcgagcagccccggtgcccgtcgtggtgagcagcgccgacgcggacgcggcccgcccgctgcgggggctgctcaagtctccgcgcggggccgacgagccagaggacagcgagctggagaggaagcgcaagatggtctccttccacggggacgtgaccgtctacctcttcgaccaggagacgccaaccaacgagctgagcgtccaggccccccccgagggggacacggacccgtcaacgcctccagcgcccccgacacctccccaccccgccacccccggagatgggtttcccagcaacgacagcggctttggaggcagtttcgagtgggcggaggatttccccctcctcccccctccaggccccccgctgtgcttctcccgcttctccgtctcgcctgcgctggagaccccggggccacccgcccgggcccccgacgcccggcccgcaggccccgtggagaattgattccccgaagacccgaccccgctgcaccctcagaagaggggttgagaatggaatcctctgtggatgacggcgccactgccaccaccgcagacgccgcctctggggaggcccccgaggctgggccctccccctcccactcccctaccatgtgccaaacgggaggccccgggcccccgccccccagccccccagatggctcccctgacccccctgaccccctcggagccaaatgaggcaggaatccccccgcccctccatagagagccgcctttctcggaactgaactgaactcttttgggcctggagcccctcgacacagcggaggtccctcctcacccactcctggcccaagacaggggccgcaggcttcggggacccggaccccccatttcgcgtctcccctttccctccccagcccggcccctggaggggcctctggttcaaaccttcgcgtggcattttcacattatttaaaaaagacaaaaacaactttttggagg

SEQ ID NO: 14 - protein, 1460 aa, Homo sapiens
MPAPGALILLAAVSASGCLASPAHPDGFALGRAPLAPPYAVVLISCSGLLAFIFLLLTCLCCKRGDVGFKEFENPEGEDCSGEYTPPAEETSSSQSLPDVYILPLAEVSLPMPAPQPSHSDMTTPLGLSRQHLSYLQEIGSGWFGKVILGEIFSDYTPAQVVVKELRASAGPLEQRKFISEAQPYRSLQHPNVLQCLGLCVETLPFLLIMEFCQLGDLKRYLRAQRPPEGLSPELPPRDLRTLQRMGLEIARGLAHLHSHNYVHSDLALRNCLLTSDLTVRIGDYGLAHSNYKEDYYLTPERLWIPLRWAAPELLGELHGTFMVVDQSRESNIWSLGVTLWELFEFGAQPYRHLSDEEVLAFVVRQQHVKLARPRLKLPYADYWYDILQSCWRPPAQRPSASDLQLQLTYLLSERPPRPPPPPPPPRDGPFPWPWPPAHSAPRPGTLSSPFPLLDGFPGADPDDVLTVTESSRGLNLECLWEKARRGAGRGGGAPAWQPASAPPAPHANPSNPFYEALSTPSVLPVISARSPSVSSEYYIRLEEHGSPPEPLFPNDWDPLDPGVPAPQAPQAPSEVPQLVSETWASPLFPAPRPFPAQSSASGSFLLSGWDPEGRGAGETLAGDPAEVLGERGTAPWVEEEEEEEEGSSPGEDSSSLGGGPSRRGPLPCPLCSREGACSCLPLERGDAVAGWGGHPALGCPHPPEDDSSLRAERGSLADLPMAPPASAPPEFLDPLMGAAAPQYPGRGPPPAPPPPPPPPRAPADPAASPDPPSAVASPGSGLSSPGPKPGDSGYETETPFSPEGAFPGGGAAEEEGVPRPRAPPEPPDPGAPRPPPDPGPLPLPGPREKPTFVVQVSTEQLLMSLREDVTRNLLGEKGATARETGPRKAGRGPGNREKVPGLNRDPTVLGNGKQAPSLSLPVNGVTVLENGDQRAPGIEEKAAENGALGSPEREEKVLENGELTPPRREEKALENGELRSPEAGEKVLVNGGLTPPKSEDKVSENGGLRFPRNTERPPETGPWRAPGPWEKTPESWGPAPTIGEPAPETSLERAPAPSAVVSSRNGGETAPGPLGPAPKNGTLEPGTERRAPETGGAPRAPGAGRLDLGSGGRAPVGTGTAPGGGPGSGVDAKAGWVDNTRPQPPPPPLPPPPEAQPRRLEPAPPRARPEVAPEGEPGAPDSRAGGDTALSGDGDPPKPERKGPEMPRLFLDLGPPQGNSEQIKARLSRLSLALPPLTLTPFPGPGPRRPPWEGADAGAAGGEAGGAGAPGPAEEDGEDEDEDEEEDEEAAAPGAAAGPRGPGRARAAPVPVVVSSADADAARPLRGLLKSPRGADEPEDSELERKRKMVSFHGDVTVYLFDQETPTNELSVQAPPEGDTDPSTPPAPPTPPHPATPGDGFPSNDSGFGGSFEWAEDFPLLPPPGPPLCFSRFSVSPALETPGPPARAPDARPAGPVEN